(19) United States
(12) Patent Application Publication
(10) Pub. No.: US 2014/0056929 A1
(43) Pub. Date: Feb. 27, 2014

(54) IMMUNOMODULATION BY CONTROLLING INTERFERON-GAMMA LEVELS WITH THE LONG NON-CODING RNA NeST

(71) Applicant: The Board of Trustees of the Leland Stanford Junior University, Palo Alto, CA (US)

(72) Inventors: Karla A. Kirkegaard, Palo Alto, CA (US); Michel Brahic, Stanford, CA (US); J. Antonio Gomez, San Mateo, CA (US); Howard Yuan-Hao Chang, Stanford, CA (US)

(73) Assignee: The Board of Trustees of the Leland Stanford Junior University, Palo Alto, CA (US)

(21) Appl. No.: 13/974,981

(22) Filed: Aug. 23, 2013

Related U.S. Application Data
(60) Provisional application No. 61/692,663, filed on Aug. 23, 2012.

(51) Int. Cl. A61K 31/7105

(52) U.S. Cl. CPC A61K 31/7105
USPC 424/184.1; 424/278.1

(57) ABSTRACT

Compositions and methods of modulating an immune response by controlling levels of interferon-gamma (IFN-γ) production by leukocytes are disclosed. Adjustment of IFN-γ levels is achieved by increasing or decreasing the activity of NeST (nettoie Salmonella pas Theiler's [cleanup Salmonella not Theiler's]), a long non-coding RNA that induces expression of IFN-γ. In particular, the invention relates to the use of NeST and inhibitors of NeST to modulate levels of IFN-γ for treatment of inflammatory conditions, autoimmune diseases, infectious diseases, immunodeficiency, and cancer.

### CROSS-REFERENCE TO RELATED APPLICATION

This application claims benefit under 35 U.S.C. §119(e) of provisional application 61/692,663, filed Aug. 23, 2012, which application is hereby incorporated by reference in its entirety.

### STATEMENT REGARDING FEDERALLY SPONSORED RESEARCH OR DEVELOPMENT

This invention was made with Government support under contract OD000827 awarded by the National Institutes of Health. The Government has certain rights in this invention.

### TECHNICAL FIELD

The present invention pertains generally to immunomodulation by interferon-gamma (IFN-γ). In particular, the invention relates to methods of modulating an immune response with NeST, a long non-coding RNA that regulates expression of IFN-γ. The invention further relates to methods of treating inflammatory, autoimmune, and infectious diseases, immunodeficiency, and cancer by controlling levels of NeST.

### BACKGROUND

Mammalian genomes are more pervasively transcribed than previously expected (Bertone et al. (2004) Science 306:2242-2246; Carninci et al. (2005) Science 309:1559-1563; Calin et al. (2007) Cancer Cell 12: 215-229; and Carninci (2008) Nat. Cell Biol. 10:1023-1024). In addition to the protein-coding regions of genes, much of the genome is transcribed as non-coding RNAs (ncRNAs). These non-coding genomic transcripts include many different types of small regulatory ncRNAs and long ncRNAs (lncRNAs).

Bioinformatic analysis of the chromatin marks in intergenic DNA regions and of expressed sequence tags (ESTs) predicts the existence of more than 5,000 long noncoding RNAs (lncRNAs) in the human genome (Guttman et al. (2009) Nature 458:223-227; Khalil et al. (2009) Proc. Natl. Acad. Sci. USA 106:11667-11672; Qureshi et al. (2010) Brain Res. 1338:20-35). Long ncRNAs vary in length from several hundred bases to tens of kilobases and may be located separate from protein coding genes (long intergenic ncRNAs or lincRNAs), or reside near or within protein coding genes (Guttman et al. (2009) Nature 458:223-227; Katayama et al. (2005) Science 309:1564-1566). It is currently unknown how many of these RNAs are functional. In a few well-studied cases, lncRNAs such as AIR, XIST, and HOTAIR have been shown to operate at the transcriptional level by binding to proteins in histone-modifying complexes and targeting them to particular genes (Chu et al. (2011) Mol. Cell. 44:667-678; Jeon and Lee (2011) Cell 146:119-133; Nagano et al. (2008) Science 322:1717-1720; Wang and Chang (2011) Mol. Cell. 43:904-914).

NeST, formally known as Tmevpg1, is an lncRNA located adjacent to the interferon (IFN)-γ-encoding gene in both mice (Ifng) and humans (IFNG). NeST was originally identified as a candidate gene in a susceptibility locus for Theiler's virus (NeST stands for nettoie Salmonella pas Theiler's [cleanup Salmonella not Theiler's]). In both mouse and human genomes, NeST RNA is encoded on the DNA strand opposite to that coding for IFN-γ, and the two genes are transcribed convergently (FIG. 1A). In the mouse, NeST RNA contains six exons spread over a 45 kilobase (kb) region (Vigneau et al. (2001) Genomics 78:206-213; Vigneau et al. (2003) J. Virol. 77:5632-5638). The most abundant splice variant is 914 nucleotides long, expressed in CD4⁺ T cells, CD8⁺ T cells, and natural killer cells, and contains no AUG codons in translational contexts that appear functional.

LncRNAs may potentially be useful therapeutically; however, the functions of only a few lncRNAs have been studied in detail, and many more functional lncRNAs have yet to be discovered. Thus, there remains a need in the art for identifying and characterizing lncRNAs that can be used in developing therapeutics.

### SUMMARY

The invention relates to compositions and methods of modulating an immune response by controlling levels of IFN-γ production by leukocytes. Adjustment of IFN-γ levels is achieved by increasing or decreasing the activity of NeST (nettoie Salmonella pas Theiler's [cleanup Salmonella not Theiler's]), a long non-coding RNA that induces expression of IFN-γ. In particular, the invention relates to the use of NeST and inhibitors of NeST, or recombinant nucleic acids encoding NeST or inhibitors of NeST to modulate levels of IFN-γ for treatment of inflammatory conditions, autoimmune diseases, infections by pathogens (e.g., viruses, bacteria, fungi, and protists or other eukaryotic parasites), immunodeficiency, and cancer.

In one aspect, the invention includes a method of modulating an immune response in a subject, the method comprising administering a therapeutically effective amount of NeST to the subject. Administering NeST increases production of IFN-γ by leukocytes (e.g., T cells, natural killer cells, myeloid cells, dendritic cells, and macrophages), whereby CD4+ helper T (Th) cells, CD8+ cytotoxic T cells, and macrophages are activated in the subject. In certain embodiments, NeST or a recombinant polynucleotide comprising a promoter operably linked to a polynucleotide encoding NeST is administered to the subject. The recombinant polynucleotide may comprise an expression vector, for example, a bacterial plasmid vector or a viral expression vector, such as, but not limited to, an adenovirus, retrovirus (e.g., γ-retrovirus and lentivirus), poxvirus, adeno-associated virus, baculovirus, or herpes simplex virus vector.

In certain embodiments, the subject has an infectious disease caused by a pathogen. For example, the infectious disease may be caused by a virus (e.g., influenza virus, respiratory syncytial virus, hepatitis virus B, hepatitis virus C, herpes virus, papilloma virus, and human immunodeficiency virus). In another example, the infectious disease is caused by a bacterial infection (e.g., tuberculosis, listeriosis, diphtheria, food poisoning, or sepsis). In one embodiment, the bacterial infection is antibiotic-resistant. In another example, the infectious disease is caused by a fungal infection (e.g., aspergillosis, blastomycosis, or candidosis). In yet another example, the infectious disease is caused by a parasite (e.g., malaria, leishmaniasis, toxoplasmosis, schistosomiasis, and clonorchiasis). In a further embodiment, the subject has cancer, tumors, or abnormal cells or tissue.

In another embodiment, the method further comprises administering a vaccine to the subject. The vaccine may be administered concurrently with NeST, for example, to augment the immune response to a pathogen or cancerous cells.

In another embodiment, NeST is administered to a subject who is immunodeficient or immunocompromised in order to increase an immune response in the subject.

In another aspect, the invention includes a method of modulating an immune response in a subject, the method comprising administering a therapeutically effective amount of a NeST inhibitor to the subject, wherein the NeST inhibitor reduces inflammation in the subject. Exemplary NeST inhibitors include antisense oligonucleotides, inhibitory RNA molecules, such as miRNAs, siRNAs, piRNAs, and snRNAs, and ribozymes. In certain embodiments, the NeST inhibitor is a recombinant polynucleotide comprising a promoter operably linked to a polynucleotide encoding an inhibitor of NeST. The recombinant polynucleotide may comprise an expression vector, for example, a bacterial plasmid vector or a viral expression vector, such as, but not limited to, an adenovirus, retrovirus (e.g., γ-retrovirus and lentivirus), poxvirus, adeno-associated virus, baculovirus, or herpes simplex virus vector.

In certain embodiments, the subject has an inflammatory condition or an autoimmune disorder, such as, but not limited to multiple sclerosis, rheumatoid arthritis, stomatitis, lupus erythematosus, ischemic heart disease, atherosclerosis, cancer, fibrosis, autoimmune thyroid disease (AITD), inflammatory bowel disease, inflammatory myopathy, giant cell arteritis (GCA), asthma, allergy, Parkinson's disease, or Alzheimer's disease. In another embodiment, the subject has damaged tissue or a wound.

In another aspect, the invention includes a method of increasing production of IFN-γ by leukocytes (e.g., T cells, natural killer cells, myeloid cells, dendritic cells, and macrophages) in a subject, the method comprising administering an effective amount of NeST or a vector encoding NeST to the subject.

In another aspect, the invention includes a method of decreasing production of IFN-γ by leukocytes in a subject, the method comprising administering an effective amount of a NeST inhibitor or a vector encoding a NeST inhibitor to the subject.

In another aspect, the invention includes a method for treating an infectious disease comprising administering to a subject in need thereof a therapeutically effective amount of NeST.

In another aspect, the invention includes a method for treating cancer comprising administering to a subject in need thereof a therapeutically effective amount of NeST.

In another aspect, the invention includes a method for treating an inflammatory condition or autoimmune disorder comprising administering to a subject in need thereof a therapeutically effective amount of at least one NeST inhibitor.

In another aspect, the invention includes a method for inhibiting NeST in a subject comprising administering an effective amount of a NeST inhibitor to the subject.

In yet another aspect, the invention provides kits comprising compositions containing NeST or at least one NeST inhibitor, or recombinant nucleic acids encoding them. The kit may also include one or more transfection reagents to facilitate delivery of oligonucleotides or polynucleotides to cells. The kit may further contain means for administering NeST or a NeST inhibitor to a subject and instructions for treating inflammatory conditions, autoimmune diseases, infections, immunodeficiency, or cancer.

These and other embodiments of the subject invention will readily occur to those of skill in the art in view of the disclosure herein.

### BRIEF DESCRIPTION OF THE FIGURES

FIGS. 1A-1D show the genotypes of the parental and congenic strains used to investigate NeST RNA and the Tmevp3 locus on murine chromosome 10. FIG. 1A shows a schematic of the NeST-encoding genes in mouse chromosome 10 and human chromosome 12. The bars represent exons, and arrows indicate the direction of transcription. NeST (previously termed Tmevpg1) is adjacent to both murine Ifng and human IFNG (Vigneau et al. (2003), supra). The major transcript, shown in light gray, encodes six exons. In both mice and humans, the NeST and IFN-γ-encoding transcripts are convergently synthesized; in humans, the transcribed regions overlap. FIG. 1B shows a diagram of the Tmevp3 locus on murine chromosome 10, as defined by the differential ability to clear persistent infection by Theiler's virus observed between SJL/J and B10.S mice. The SJL/J.Tmevp3^{B10.S} line (previously termed C2; Vigneau et al. (2003), supra) is congenic with SJL/J except for the region shown, from microsatellite marker D10Mit74 to the interval between D10Mit180 and D10Mit233. The B10.S.Tmevp3^{SJL/J} strain is congenic with B10.S except for the region shown, between the D10Mit151/D10Mit271 interval and the D10Mit233/D10Mit73 interval. The Theiler's virus (TMEV) persistence and clearance phenotypes and Nramp1 alleles for all four strains are listed. FIG. 1C shows finer mapping of the polymorphic regions of the congenic lines. The X axis indicates the nucleotide number on mouse chromosome 10 (NCBI37/mm9). The introgressed region of SJL/J in B10.S.Tmevp3^{SJL/J} is up to 1.6×10⁷ base pairs (bp) (top), whereas the introgression in SJL/J.Tmevp3^{B10.S} is approximately 5.5×10⁵ bp (middle and bottom). Each bar displays the number of SNPs in the window size indicated. The most polymorphic region maps to the Tmevp3 locus and coincides with the region of introgression in SJL/J.Tmevp3^{B10.S}; see Table 1 for lists of all genetic differences between the two Tmepv3 alleles. The physical locations and direction of transcription of the murine NeST, Ifng, Il22, and Mdm1 genes are indicated by arrows. FIG. 1D shows NeST RNA expression in CD3⁺ T cells. The abundance of NeST RNA in CD3⁺ splenocytes from B10.S mice and B10.S.Tmevp3^{SJL/J} was determined by preparing total cellular RNA and determining the amount of RNA per cell using qRT-PCR and standard curves of transcribed RNAs. The threshold of detection was 0.005 molecules of NeST RNA per cell. Mean values are shown with SE.

FIGS. 2A-2F show the effect of the Tmevp3 locus on salmonella pathogenesis. FIGS. 2A and 2B show that strains SJL/J and SJL/J.Tmevp3B10.S were inoculated by oral (FIG. 2A) and intraperitoneal (FIG. 2B) routes with S. enterica Typhimurium. The Nramp1^{+/+} alleles expressed by SJL/J and SJL/J.Tmevp3B10.S mice render them relatively resistant to Salmonella infection. FIGS. 2C and 2D show strains B10.S and B10.S.Tmevp3^{SJL/J}, which were also inoculated by oral (FIG. 2C) and intraperitoneal (FIG. 2D) routes with S. enterica Typhimurium at the dosages indicated, and mortality was monitored. The Nramp1^{169Asp/169Asp} alleles render these mice highly sensitive to Salmonella pathogenesis. In both backgrounds, the SJL/J allele of the Tmevp3 locus reduced mortality after oral inoculation. Statistical significance was determined by the log rank test. FIG. 2E shows B10.S and B10.S.Tmevp3^{SJL/J} strains, which were orally inoculated with S. enterica Typhimurium at 10⁶ CFU/mouse. Bacteria were monitored in spleen and feces at the indicated days. FIG. 2F shows intracellular bacterial growth, which was monitored ex vivo in bone-marrow-derived macrophages from B10.S and B10.S.Tmevp3^{SJL/J} mice. Lines represent the mean of triplicate experiments, and statistical significance was determined using a Student's t test.

FIGS. 3A-3C show the effects of transgenically expressed NeST RNA on salmonella pathogenesis. FIG. 3A shows a schematic of transgenes introduced into B10.S mice. SJL/J NeST cDNA (dark gray) and B10.S NeST cDNA (light gray) were cloned downstream of a CD4⁺ and CD8⁺ T cell-specific promoter. The promoter-NeST transgene fragments were used to construct transgenic mouse lines in the B10.S background. FIG. 3B shows the abundance of NeST RNA that was measured in CD8⁺ splenocytes from B10.S mice congenic for the SJL/J-derived Tmevp3 locus (B10.S.Tmevp3^{SJL/J}), B10.S mice, B10.S mice containing the SJL/J NeST transgene (B10.S.NeST^{SJL/J}), and B10.S mice containing the B10.S NeST transgene (B10.S.NeST^{B10.S}). The amount of RNA per cell was determined using qRT-PCR; in vitro transcribed NeST RNA was used to construct standard curves. Mean values are shown with SE. FIG. 3C shows the results with B10.S, B10.S.Tmevp3^{SJL/J}, B10.S.NeST^{SJL/J}, and B10.S.NeST^{B10.S} mice, which were orally inoculated with S. enterica Typhimurium at the dosages indicated, and mortality was monitored. All experiments with the 10⁷ CFU/mouse were performed at the same time; the B10.S control is shown in these panels for clarity. Statistical significance was determined by the log rank test.

FIGS. 4A-4C show the effect of NeST RNA on Theiler's virus persistence. B10.S mice, B10.S mice congenic for the Tmevp3 locus from SJL/J mice (B10.S.Tmevp3SJL/J), and B10.S mice containing the B10.S NeST transgene (B10.S.NeSTB10.S) were inoculated by intracranial injections of 10⁷ plaque-forming units (pfu) of Theiler's virus. FIGS. 4A and 4B show results from spinal cords, which were harvested at 7 days (FIG. 4A) and 57 days (FIG. 4B) post inoculation, and viral load was measured by plaque assay on BHK-21 cell monolayers. FIG. C4 shows the abundance of viral RNA in spinal cords from B10.S, B10.S.Tmevp3^{SJL/J} and B10.S.NeSTB10.S mice, which was determined by preparing total cellular RNA from homogenized tissue and determining the amount of viral RNA per gram of tissue using qRT-PCR. TMEV RNA was transcribed from cDNA-containing plasmid to construct standard curves. Means and standard error (SE) are shown.

FIGS. 5A-5C show the effect of the Tmevp3 locus and transgenically expressed NeST RNA on cytokine expression by T cell subsets. FIGS. 5A and 5B show results from splenic (FIG. 5A) CD4⁺ and (FIG. 5B) CD8⁺ T cells, which were isolated from three B10.S (black circles) and three B10.S.Tmevp3^{SJL/J} (white circles) mice and stimulated ex vivo with PMA and ionomycin. The abundances of IFN-γ and IL-22 protein secreted were determined by ELISA from supernatants collected at the indicated times. Means and SE are indicated for each time point. Statistical significance was determined using a two-way ANOVA test; asterisks denote values that differ significantly between T cells derived from B10.S and T cells derived from B10.S.Tmevp3^{SJL/J} mice. FIG. 5C shows results from splenic CD8⁺ T cells, which were isolated from B10.S (black), B10.S.NeST^{SJL/J} (dark gray), and B10.S.NeST^{B10.S} (light) mice and stimulated ex vivo with PMA and ionomycin. The abundance of secreted IFN-γ was determined by ELISA. Asterisks and p values refer to the comparisons between T cells derived from B10.S and T cells derived from each transgenic line.

FIGS. 6A-6C show NeST RNA localization and IFN-γ trans activation. FIG. 6A shows nuclear and cytoplasmic RNA from CD8 T cells from B10.S.Tmevp3^{SJL/J}, B10.S.NeST^{B10.S}, and B10.S.NeST^{SJL/J} mice, which were fractionated by differential centrifugation (Huarte et al. (2010) Cell 142:409-419). NeST RNA, unspliced actin RNA (nuclear), and spliced actin RNA (cytoplasmic) from the nuclear and cytoplasmic fractions were assessed by RT-PCR and gel electrophoresis. FIG. 6B shows quantitation of expression ratios of IFN-γ mRNA from the B10.S and the B10.S.Tmevp3^{SJL/S} alleles. A natural SNP in the IFN-γ mRNA (coordinate 117882772; see Table 1) was amplified by RT-PCR (top and left panel). CDNAs from B10.S and B10.S.Tmevp3^{SJL/J} were subjected to a B10.S allele-specific TaqI restriction digest (bottom, left) and fragments were analyzed by gel electrophoresis. FIG. 6C shows results from splenic CD8 T cells, which were isolated from two B10.S 3×B10.S.Tmevp3^{SJL/J} heterozygous mice and stimulated with PMA and ionomycin. The proportion of B10.S and B10.S.Tmevp3^{SJL/J}-derived IFN-γ mRNA was determined by densitometry of the allele-specific restriction fragments. Mixtures of in vitro transcribed RNAs at 1:10 and 1:1 ratios were used as controls.

FIGS. 7A-7C shows NeST RNA's physical association with WDR5 protein and its effect on histone 3 lysine 4 trimethylation at the Ifng locus. FIG. 7A shows RNA preparation from 293T cells that were cotransfected with FLAG-tagged WDR5 cDNA and either B10.S-derived NeST cDNA (light gray) or SJL/J-derived NeST cDNA (dark gray) and analyzed after immunoprecipitation with either anti-FLAG antibodies or anti-immunoglobulin G (anti-IgG) control antibodies. NeST RNA retrieval was determined by measuring RNA input levels normalized to glyceraldehyde 3-phosphate dehydrogenase (GAPDH; bottom left panel). Specific RNA retrieval was determined by subtracting NeST RNA retrieval with anti-IgG antibodies from the retrieval with anti-FLAG antibodies, followed by normalization to the amount of input RNA. Immunoblot analysis (bottom right panel) confirmed FLAG-WDR5 expression following transfection and the specificity of the anti-FLAG and anti-IgG antibodies. FIG. 7B shows IFN-γ production and H3K4me3 occupancy in spleen following immune challenge. B10.S, B10.S.NeST^{B10.S}, and B10.S.NeST^{SJL/J} mice were injected intraperitoneally with 50 μg of LPS, and spleens were dissected 4 and 6 hours later. The abundance of IFN-γ protein was determined by ELISA in tissue homogenates (top panel) and the occupancy of histone 3 lysine 4 trimethylation at the Ifng gene was determined by ChIP-qPCR analysis (bottom panel). A schematic diagram of the positions of primers used for H3K4me3 is shown. Specific DNA retrieval was measured by normalization to the amount of input DNA and ChIP signal at GAPDH loci. FIG. 7C shows ChIP-qPCR analysis of H3K4me3 at the Ifng locus in CD8 T cells from B10.S and B10.S.NeST^{SJL/J} transgenic mice. CD8 T cells, which were isolated from four B10.S and four B10.S.NeST^{SJL/J} mice, and stimulated ex vivo with PMA and ionomycin. Occupancy of H3K4me3 at the Ifng gene was assayed 24 hours after stimulation by ChIP-qPCR at four different regions. For all pooled data, means and SE are shown.

FIG. 8 shows the lethal inflammatory phenotype is linked to the Tmevp3 locus (related to FIG. 2). In two independent experiments, strains B10.S and B10.S.Tmevp3^{SJL/J} were injected intraperitoneally with 100 mg of lipopolysaccharides (LPS) and mortality was monitored. The SJL/J allele of the Tmevp3 locus reduced mortality. Statistical significance was determined by the logrank test.

FIGS. 9A-9C show cytokine and NeST RNA expression in CD4⁺ and CD8⁺ T cells (related to FIG. 5). FIGS. 9A and 9B show results from splenic (FIG. 9A) CD4 and (FIG. 9B) CD8 T cells, which were isolated from three B10.5 (black circles) and three B10.S.Tmevp3^{SJL/J} (white circles) mice and stimulated ex vivo with PMA and ionomycin. The abundance of IFN-γ and NeST RNA per cell was determined using quantitative RT-PCR. Means and standard error are indicated for each time point. Statistical significance was determined using a two-way ANOVA test; asterisks denote those values that differ significantly between T cells derived from B10.S and T cells derived from B10.S.Tmevp3^{SJL/J} mice. FIG. 9C shows results from CD8 T cells from three B10.S (black) and two B10.S.Tmevp3^{SJL/J} (white) mice, which were stimulated with PMA and ionomycin ex vivo. The abundance of 27 different cytokines was assayed 24 hours later with the Luminex assay. Significant differences in expression were observed for IFN-γ (p=0.0322), IL-2 (p=0.0265), IL-13 (p=0.0314), IL-17 (p=0.0469), RANTES (p=0.0417), and TNF-α (p=0.0058).

### DETAILED DESCRIPTION

The practice of the present invention will employ, unless otherwise indicated, conventional methods of pharmacology, chemistry, biochemistry, recombinant DNA techniques and immunology, within the skill of the art. Such techniques are explained fully in the literature. See, e.g., Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (3^{rd} Edition, 2001); Methods In Enzymology (S. Colowick and N. Kaplan eds., Academic Press, Inc.).

All publications, patents and patent applications cited herein, whether supra or infra, are hereby incorporated by reference in their entireties.

### I. DEFINITIONS

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “a lncRNA” includes a mixture of two or more lncRNAs, and the like.

The term “about,” particularly in reference to a given quantity, is meant to encompass deviations of plus or minus five percent.

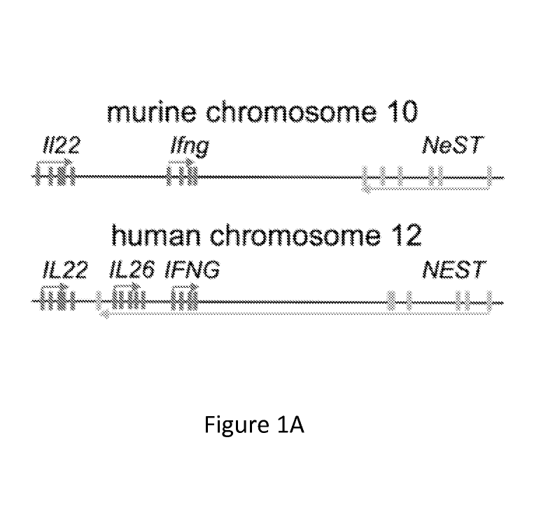

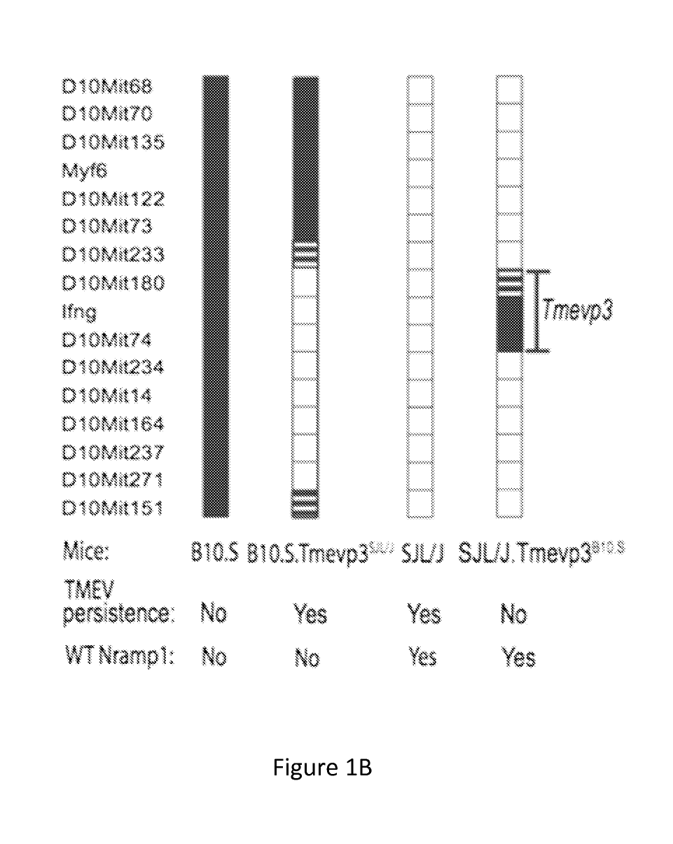

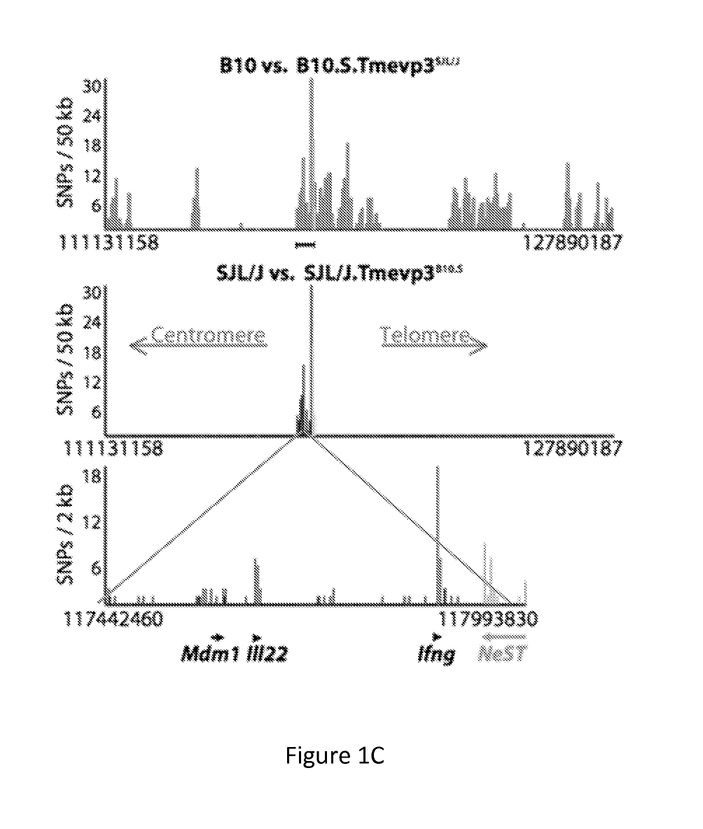

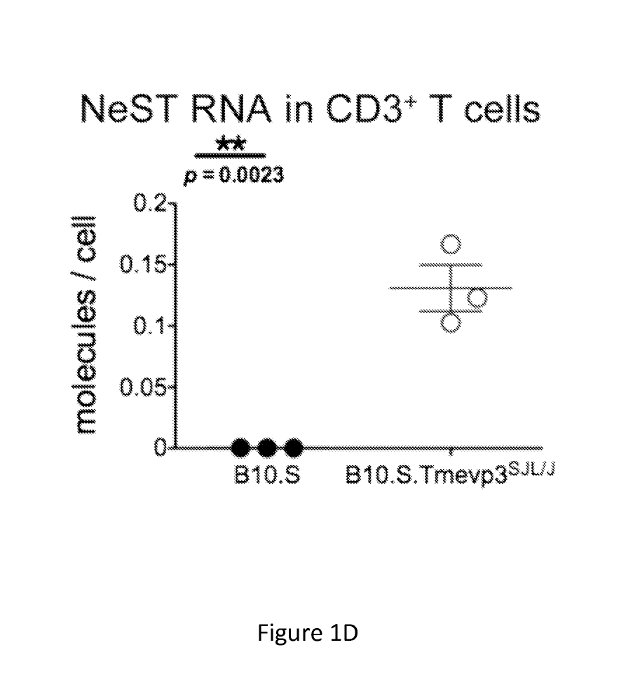

“NeST” refers to Nettoie Salmonella pas Theiler's (clean up Salmonella not Theiler's), also known as Tmevpg1, a long non-coding RNA transcript located adjacent to the interferon IFN-γ-encoding gene (see, e.g., FIG. 1A and Vigneau et al. (2001) Genomics 78:206-213; Collier et al. (2012) J. Immunol. 189:2084-2088; herein incorporated by reference). Human NeST is located on chromosome 12q15. A representative human sequence of NeST is shown in SEQ ID NO:1.

The term “immunomodulatory” or “modulating an immune response” as used herein includes immunostimulatory as well as immunosuppressive effects. Immunomodulation, for example, by NeST or a NeST inhibitor may cause an increase or decrease in IFN-γ production, respectively, in an individual treated in accordance with the methods of the invention as compared to the absence of treatment. The level of IFN-γ, secreted by leukocytes (e.g., T cells, natural killer cells, myeloid cells, dendritic cells, and macrophages), in turn modulates innate and cellular immune responses by controlling activation of CD4+ helper T (Th) cells, CD8+ cytotoxic T cells, macrophages, and natural killer cells.

The terms “microRNA,” “miRNA,” and MiR” are interchangeable and refer to endogenous or artificial non-coding RNAs that are capable of regulating gene expression. It is believed that miRNAs function via RNA interference. When used herein in the context of inactivation, the use of the term microRNAs is intended to include also long non-coding RNAs, piRNAs, siRNAs, and the like. Endogenous (e.g., naturally occurring) miRNAs are typically expressed from RNA polymerase II promoters and are generated from a larger transcript.

The terms “siRNA” and “short interfering RNA” are interchangeable and refer to single-stranded or double-stranded RNA molecules that are capable of inducing RNA interference. SiRNA molecules typically have a duplex region that is between 18 and 30 base pairs in length.

The terms “piRNA” and “Piwi-interacting RNA” are interchangeable and refer to a class of small RNAs involved in gene silencing. PiRNA molecules typically are between 26 and 31 nucleotides in length.

The terms “snRNA” and “small nuclear RNA” are interchangeable and refer to a class of small RNAs involved in a variety of processes including RNA splicing and regulation of transcription factors. The subclass of small nucleolar RNAs (snoRNAs) is also included. The term is also intended to include artificial snRNAs, such as antisense derivatives of snRNAs comprising antisense sequences directed against the lncRNA, NeST.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, microRNA, DNA:RNA hybrids, and hybrids between PNAs and DNA or RNA, and also include known types of modifications, for example, labels which are known in the art, methylation, “caps,” substitution of one or more of the naturally occurring nucleotides with an analog (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. The term also includes locked nucleic acids (e.g., comprising a ribonucleotide that has a methylene bridge between the 2′-oxygen atom and the 4′-carbon atom). See, for example, Kurreck et al. (2002) Nucleic Acids Res. 30: 1911-1918; Elayadi et al. (2001) Curr. Opinion Invest. Drugs 2: 558-561; Orum et al. (2001) Curr. Opinion Mol. Ther. 3: 239-243; Koshkin et al. (1998) Tetrahedron 54: 3607-3630; Obika et al. (1998) Tetrahedron Lett. 39: 5401-5404.

The term “homologous region” refers to a region of a nucleic acid with homology to another nucleic acid region. Thus, whether a “homologous region” is present in a nucleic acid molecule is determined with reference to another nucleic acid region in the same or a different molecule. Further, since a nucleic acid is often double-stranded, the term “homologous, region,” as used herein, refers to the ability of nucleic acid molecules to hybridize to each other. For example, a single-stranded nucleic acid molecule can have two homologous regions which are capable of hybridizing to each other. Thus, the term “homologous region” includes nucleic acid segments with complementary sequence. Homologous regions may vary in length, but will typically be between 4 and 40 nucleotides (e.g., from about 4 to about 40, from about 5 to about 40, from about 5 to about 35, from about 5 to about 30, from about 5 to about 20, from about 6 to about 30, from about 6 to about 25, from about 6 to about 15, from about 7 to about 18, from about 8 to about 20, from about 8 to about 15, etc.).

The term “complementary” and “complementarity” are interchangeable and refer to the ability of polynucleotides to form base pairs with one another. Base pairs are typically formed by hydrogen bonds between nucleotide units in antiparallel polynucleotide strands or regions. Complementary polynucleotide strands or regions can base pair in the Watson-Crick manner (e.g., A to T, A to U, C to G). 100% complementary refers to the situation in which each nucleotide unit of one polynucleotide strand or region can hydrogen bond with each nucleotide unit of a second polynucleotide strand or region. Less than perfect complementarity refers to the situation in which some, but not all, nucleotide units of two strands or two regions can hydrogen bond with each other and can be expressed as a percentage.

A “target site” or “target sequence” is the nucleic acid sequence recognized (i.e., sufficiently complementary for hybridization) by an antisense oligonucleotide or inhibitory RNA molecule.

The term “hairpin” and “stem-loop” can be used interchangeably and refer to stem-loop structures. The stem results from two sequences of nucleic acid or modified nucleic acid annealing together to generate a duplex. The loop lies between the two strands comprising the stem.

The term “loop” refers to the part of the stem-loop between the two homologous regions (the stem) that can loop around to allow base-pairing of the two homologous regions. The loop can be composed of nucleic acid (e.g., DNA or RNA) or non-nucleic acid material(s), referred to herein as nucleotide or non-nucleotide loops. A non-nucleotide loop can also be situated at the end of a nucleotide molecule with or without a stem structure.

“Inhibition of gene expression” refers to the absence (or observable decrease) in the level of protein and/or RNA product from a target gene. “Specificity” refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed.

Depending on the assay, quantitation of the amount of gene expression allows one to determine a degree of inhibition which is greater than 10%, 33%, 50%, 90%, 95% or 99% as compared to a cell not treated with the inhibitory agent. Lower doses of the administered inhibitory agent and longer times after administration of inhibitory agent may result in inhibition in a smaller fraction of cells (e.g., at least 10%, 20%, 50%, 75%, 90%, or 95% of targeted cells). Quantitation of gene expression in a cell may show similar amounts of inhibition at the level of accumulation of target RNA or translation of target protein. As an example, the efficiency of inhibition may be determined by assessing the amount of gene product in the cell: RNA may be detected with a hybridization probe having a nucleotide sequence outside the region used for the inhibitory, or translated polypeptide may be detected with an antibody raised against the polypeptide sequence of that region.

“Administering” a nucleic acid, such as a microRNA, siRNA, piRNA, snRNA, antisense nucleic acid, or lncRNA to a cell comprises transducing, transfecting, electroporating, translocating, fusing, phagocytosing, shooting or ballistic methods, etc., i.e., any means by which a nucleic acid can be transported across a cell membrane.

The term “transfection” is used to refer to the uptake of foreign DNA or RNA by a cell. A cell has been “transfected” when exogenous DNA or RNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA or RNA moieties into suitable host cells. The term refers to both stable and transient uptake of the genetic material, and includes uptake, for example, of microRNA, siRNA, piRNA, lncRNA, or antisense nucleic acids.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

As used herein, the term “pathogen” or “parasite” or “microbe” refers to any virus or organism that spends at least part of its life cycle or reproduces within a host. Intracellular pathogens include viruses (e.g., influenza virus, respiratory syncytial virus, hepatitis virus B, hepatitis virus C, herpes virus, papilloma virus, and human immunodeficiency virus), bacteria (e.g., Listeria, Mycobacteria (e.g., Mycobacterium tuberculosis, Mycobacterium leprae), Salmonella (e.g., S. typhi), enteropathogenic Escherichia coli (EPEC), enterohaemorrhagic Escherichia coli (EHEC), Yersinia, Shigella, Chlamydia, Chlamydophila, Staphylococcus, Legionella), protozoa (e.g., Plasmodium (e.g., P. vivax, P. falciparum, P. ovale, and P. malariae), Taxoplasma, Leishmania), and fungi (e.g., Aspergillus, Blastomyces, Candida). Eukaryotic intercellular parasites include trematodes (e.g., Schistosoma, Clonorchis), hookworms (e.g., Ancylostoma duodenale and Necator americanus), and tape worms (e.g., Taenia solium, T. saginata, Diphyllobothrium spp., Hymenolepis spp., Echinococcus spp.).

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission. In particular, the terms “tumor,” “cancer” and “neoplasia” include carcinomas, such as squamous cell carcinoma, adenocarcinoma, adenosquamous carcinoma, anaplastic carcinoma, large cell carcinoma, and small cell carcinoma. These terms include, but are not limited to, breast cancer, prostate cancer, lung cancer, ovarian cancer, bladder cancer, testicular cancer, colon cancer, pancreatic cancer, gastric cancer, hepatic cancer, leukemia, lymphoma, adrenal cancer, thyroid cancer, pituitary cancer, renal cancer, brain cancer, skin cancer, head cancer, neck cancer, oral cavity cancer, tongue cancer, and throat cancer.

An “effective amount” of NeST is an amount sufficient to effect beneficial or desired results, such as an amount that increases production of IFN-γ from leukocytes. An effective amount can be administered in one or more administrations, applications, or dosages.

An “effective amount” of a NeST inhibitor (e.g., microRNA, siRNA, piRNA, snRNA, antisense nucleic acid, ribozyme, or small molecule inhibitor) is an amount sufficient to effect beneficial or desired results, such as an amount that inhibits the activity of NeST, for example by interfering with transcription of NeST, binding of NeST to WDR5, or activation of IFN-γ gene expression. An effective amount can be administered in one or more administrations, applications, or dosages.

By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Such activity can be assessed using animal models.

By “therapeutically effective dose or amount” of NeST is intended an amount that, when administered as described herein, brings about a positive therapeutic response, such as improved recovery from an infectious disease, immunodeficiency, or cancer. Improved recovery may involve activation of the immune system against pathogens or cancerous cells by raising the level of IFN-γ produced by leukocytes to provide immunoregulatory, antiviral, antiseptic, anti-tumor, or anti-metastatic activity. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

By “therapeutically effective dose or amount” of a NeST inhibitor is intended an amount that, when administered as described herein, brings about a positive therapeutic response, such as improved recovery from an inflammatory condition or autoimmune disorder. Improved recovery may include reducing inflammation associated with a disease such as, but not limited to an autoimmune disease, a cardiovascular disease, or a neurodegenerative disorder, or resulting from the inflammatory response to damaged cells or wounds. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

“Substantially purified” generally refers to isolation of a substance (compound, polynucleotide, protein, polypeptide, polypeptide composition) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

“Homology” refers to the percent identity between two polynucleotide or two polypeptide moieties. Two nucleic acid, or two polypeptide sequences are “substantially homologous” to each other when the sequences exhibit at least about 50% sequence identity, preferably at least about 75% sequence identity, more preferably at least about 80%-85% sequence identity, more preferably at least about 90% sequence identity, and most preferably at least about 95%-98% sequence identity over a defined length of the molecules. As used herein, substantially homologous also refers to sequences showing complete identity to the specified sequence.

In general, “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353 358, National biomedical Research Foundation, Washington, D.C., which adapts the local homology algorithm of Smith and Waterman Advances in Appl. Math. 2:482 489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

“Recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells”, “host cells,” “cells”, “cell lines,” “cell cultures”, and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

“Operably linked” refers to an arrangement of elements wherein the components so described are configured so as to perform their usual function. Thus, a given promoter operably linked to a coding sequence is capable of effecting the expression of the coding sequence when the proper enzymes are present. Expression is meant to include the transcription of any one or more of transcription of a microRNA, siRNA, piRNA, snRNA, lncRNA, antisense nucleic acid, or mRNA from a DNA or RNA template and can further include translation of a protein from an mRNA template. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Purified polynucleotide” refers to a polynucleotide of interest or fragment thereof which is essentially free, e.g., contains less than about 50%, preferably less than about 70%, and more preferably less than about at least 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

The terms “variant” refers to biologically active derivatives of the reference molecule that retain desired activity, such as lncRNA gene regulatory activity, RNA interference (RNAi), or transcription factor activity. In general, the term “variant” refers to molecules (e.g., lncRNAs, miRNAs, siRNAs, piRNAs, snRNAs, antisense nucleic acids, or other inhibitors of lncRNAs) having a native sequence and structure with one or more additions, substitutions (generally conservative in nature) and/or deletions, relative to the native molecule, so long as the modifications do not destroy biological activity and which are “substantially homologous” to the reference molecule. In general, the sequences of such variants will have a high degree of sequence homology to the reference sequence, e.g., sequence homology of more than 50%, generally more than 60%-70%, even more particularly 80%-85% or more, such as at least 90%-95% or more, when the two sequences are aligned.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses.

The term “derived from” is used herein to identify the original source of a molecule but is not meant to limit the method by which the molecule is made which can be, for example, by chemical synthesis or recombinant means.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) increasing survival time; (b) decreasing the risk of death due to the disease; (c) preventing the disease from occurring in a subject which may be predisposed to the disease but has not yet been diagnosed as having it; (d) inhibiting the disease, i.e., arresting its development (e.g., reducing the rate of disease progression); and (e) relieving the disease, i.e., causing regression of the disease.

The terms “subject,” “individual,” and “patient,” are used interchangeably herein and refer to any mammalian subject for whom diagnosis, prognosis, treatment, or therapy is desired, particularly humans. Other subjects may include cattle, dogs, cats, guinea pigs, rabbits, rats, mice, horses, and so on. In some cases, the methods of the invention find use in experimental animals, in veterinary application, and in the development of animal models for disease, including, but not limited to, rodents including mice, rats, and hamsters; primates, and transgenic animals.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery that an lncRNA, referred to as NeST (nettoie Salmonella pas Theiler's [cleanup Salmonella not Theiler's]), regulates expression of IFN-γ. The inventors have further shown that increasing expression of NeST correlated with higher IFN-γ production from activated CD8+ T cells, increased Theiler's virus persistence, and decreased Salmonella enterica pathogenesis. NeST RNA was found to bind WDR5, a component of the histone H3 lysine 4 methyltransferase complex, and to alter histone 3 methylation at the IFN-γ locus. Thus, NeST regulates epigenetic marking of IFN-γ-encoding chromatin, expression of IFN-γ, and susceptibility to viral and bacterial pathogens (see Example 1).

Accordingly, IFN-γ levels can be adjusted by increasing or decreasing the levels or activity of NeST. Increasing or decreasing NeST expression in vivo causes a concomitant increase or decrease in IFN-γ production, which, in turn increases or decreases innate and cellular immune responses, which can be controlled as desired. For example, many infectious diseases, in particular, those caused by intracellular pathogens (e.g., viruses, bacteria, protists, and fungi), immunodeficiency, and cancer may be treated by increasing NeST-induced IFN-γ expression levels. Conversely, decreasing NeST expression or inhibiting NeST activity reduces inflammation in vivo and, therefore, can be used to treat various inflammatory conditions and autoimmune diseases.

In order to further an understanding of the invention, a more detailed discussion is provided below regarding NeST and therapeutic uses for NeST and inhibitors of NeST in modulating innate and cellular immune responses and treating inflammatory conditions, autoimmune diseases, infections, immunodeficiency, and cancer.

A. NeST and Inhibitors

The present invention pertains generally to compositions and methods for using NeST and inhibitors thereof to modulate levels of IFN-γ for treatment of inflammatory conditions, autoimmune diseases, infections, immunodeficiency, and cancer. Immunomodulation, for example, by NeST or a NeST inhibitor may cause an increase or decrease in IFN-γ production, respectively, in an individual treated in accordance with the methods of the invention as compared to the absence of treatment. The level of IFN-γ secreted by leukocytes (e.g., T cells, natural killer cells, myeloid cells, dendritic cells, and macrophages) in turn modulates innate and cellular immune responses by controlling activation of CD4+ helper T (Th) cells, CD8+ cytotoxic T cells, macrophages, and natural killer cells.

In certain embodiments, NeST is used in the practice of the invention to increase IFN-γ levels in a subject. NeST may be synthetically or recombinantly produced and can be provided by a polynucleotide comprising the NeST sequence. The polynucleotide may comprise one or more sequences from any NeST allele capable of increasing production of IFN-γ from leukocytes (see, e.g., Example 1, Table 1). In certain embodiments, the polynucleotide comprises the sequence of SEQ ID NO:1 or a variant thereof displaying at least about 80-100% sequence identity thereto, including any percent identity within this range, such as 81, 82, 83, 84, 85, 86, 87, 88, 89, 90, 91, 92, 93, 94, 95, 96, 97, 98, 99% sequence identity thereto, wherein the polynucleotide retains NeST lncRNA biological activity (e.g., increases production of IFN-γ from leukocytes). The polynucleotide can be single stranded or double stranded and may contain one or more chemical modifications, such as, but not limited to, locked nucleic acids, peptide nucleic acids, sugar modifications, such as 2′-O-alkyl (e.g., 2′-O-methyl, 2′-O-methoxyethyl), 2′-fluoro, and 4′-thio modifications, and backbone modifications, such as one or more phosphorothioate, morpholino, or phosphonocarboxylate linkages. In one embodiment, the polynucleotide is conjugated to cholesterol. In certain embodiments, NeST is provided by a recombinant polynucleotide comprising a NeST sequence operably linked to a promoter. In certain embodiments, the expression of NeST in a cell may be increased by at least 10%, 20%, 50%, 100%, 200%, 500%, or 10-fold, 20-fold, 50-fold, or more by such a recombinant polynucleotide.

In one embodiment, the invention includes a method of modulating an immune response in a subject, the method comprising administering a therapeutically effective amount of NeST to the subject, wherein NeST is administered in an amount sufficient to increase production of IFN-γ by leukocytes (e.g., T cells, natural killer cells, myeloid cells, dendritic cells, and macrophages). A “therapeutically effective dose or amount” of NeST is intended an amount that, when administered as described herein, brings about a positive therapeutic response, such as improved recovery from an infectious disease, immunodeficiency, or cancer and which can provide immunoregulatory, antiviral, antiseptic, anti-tumor, or anti-metastatic activity.

In another aspect, an inhibitor of NeST is used in the practice of the invention. Inhibitors of NeST can include, but are not limited to, antisense oligonucleotides, inhibitory RNA molecules, such as miRNAs, siRNAs, piRNAs, and snRNAs, ribozymes, and small molecule inhibitors. Various types of inhibitors for inhibiting nucleic acid function are well known in the art. See e.g., International patent application WO/2012/018881; U.S. patent application 2011/0251261; U.S. Pat. No. 6,713,457; Kole et al. (2012) Nat. Rev. Drug Discov. 11(2):125-40; Sanghvi (2011) Curr. Protoc. Nucleic Acid Chem. Chapter 4:Unit 4.1.1-22; herein incorporated by reference in their entireties.

Inhibitors can be single stranded or double stranded polynucleotides and may contain one or more chemical modifications, such as, but not limited to, locked nucleic acids, peptide nucleic acids, sugar modifications, such as 2′-O-alkyl (e.g., 2′-O-methyl, 2′-O-methoxyethyl), 2′-fluoro, and 4′-thio modifications, and backbone modifications, such as one or more phosphorothioate, morpholino, or phosphonocarboxylate linkages. In addition, inhibitory RNA molecules may have a “tail” covalently attached to their 3′- and/or 5′-end, which may be used to stabilize the RNA inhibitory molecule or enhance cellular uptake. Such tails include, but are not limited to, intercalating groups, various kinds of reporter groups, and lipophilic groups attached to the 3′ or 5′ ends of the RNA molecules. In certain embodiments, the RNA inhibitory molecule is conjugated to cholesterol or acridine. See, for example, the following for descriptions of syntheses of 3′-cholesterol or 3′-acridine modified oligonucleotides: Gamper, H. B., Reed, M. W., Cox, T., Virosco, J. S., Adams, A. D., Gall, A., Scholler, J. K., and Meyer, R. B. (1993) Facile Preparation and Exonuclease Stability of 3′-Modified Oligodeoxynucleotides. Nucleic Acids Res. 21:145-150; and Reed, M. W., Adams, A. D., Nelson, J. S., and Meyer, R. B., Jr. (1991) Acridine and Cholesterol-Derivatized Solid Supports for Improved Synthesis of 3′-Modified Oligonucleotides. Bioconjugate Chem. 2:217-225 (1993); herein incorporated by reference in their entireties. Additional lipophilic moieties that can be used, include, but are not limited to, oleyl, retinyl, and cholesteryl residues, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O₃-(oleoyl)lithocholic acid, 0₃-(oleoyl)cholenic acid, dimethoxytrityl, or phenoxazine. Additional compounds, and methods of use, are set out in US Patent Publication Nos. 2010/0076056, 2009/0247608 and 2009/0131360; herein incorporated by reference in their entireties.

In one embodiment, inhibition of NeST function may be achieved by administering antisense oligonucleotides targeting NeST. The antisense oligonucleotides may be ribonucleotides or deoxyribonucleotides. Preferably, the antisense oligonucleotides have at least one chemical modification. Antisense oligonucleotides may be comprised of one or more “locked nucleic acids”. “Locked nucleic acids” (LNAs) are modified ribonucleotides that contain an extra bridge between the 2′ and 4′ carbons of the ribose sugar moiety resulting in a “locked” conformation that confers enhanced thermal stability to oligonucleotides containing the LNAs. Alternatively, the antisense oligonucleotides may comprise peptide nucleic acids (PNAs), which contain a peptide-based backbone rather than a sugar-phosphate backbone. The antisense oligonucleotides may contain one or more chemical modifications, including, but are not limited to, sugar modifications, such as 2′-O-alkyl (e.g. 2′-O-methyl, 2′-O-methoxyethyl), 2′-fluoro, and 4′ thio modifications, and backbone modifications, such as one or more phosphorothioate, morpholino, or phosphonocarboxylate linkages (see, for example, U.S. Pat. Nos. 6,693,187 and 7,067,641, which are herein incorporated by reference in their entireties). In some embodiments, suitable antisense oligonucleotides are 2′-O-methoxyethyl “gapmers” which contain 2′-O-methoxyethyl-modified ribonucleotides on both 5′ and 3′ ends with at least ten deoxyribonucleotides in the center. These “gapmers” are capable of triggering RNase H-dependent degradation mechanisms of RNA targets. Other modifications of antisense oligonucleotides to enhance stability and improve efficacy, such as those described in U.S. Pat. No. 6,838,283, which is herein incorporated by reference in its entirety, are known in the art and are suitable for use in the methods of the invention. Antisense oligonucleotides may comprise a sequence that is at least partially complementary to a NeST target sequence, e.g., at least about 75%, 80%, 85%, 90%, 91%, 92%, 93%, 94%, 95%, 96%, 97%, 98%, or 99% complementary to the NeST target sequence. In some embodiments, the antisense oligonucleotide may be substantially complementary to the NeST target sequence, that is at least about 95%, 96%, 97%, 98%, or 99% complementary to a target polynucleotide sequence. In one embodiment, the antisense oligonucleotide comprises a sequence that is 100% complementary to the NeST target sequence. In one embodiment, the antisense oligonucleotide targets the NeST sequence of SEQ ID NO:1.

In another embodiment, the inhibitor of NeST is an inhibitory RNA molecule (e.g., a miRNA, a siRNA, a piRNA, or a snRNA) having a single-stranded or double-stranded region that is at least partially complementary to the target sequence of NeST, e.g., about 75%, 80%, 85%, 90%, 91%, 92%, 93%, 94%, 95%, 96%, 97%, 98%, or 99% complementary to the target sequence of NeST. In some embodiments, the inhibitory RNA comprises a sequence that is substantially complementary to the target sequence of NeST, e.g., about 95%, 96%, 97%, 98%, or 99% complementary to a target polynucleotide sequence. In other embodiments, the inhibitory RNA molecule may contain a region that has 100% complementarity to the target sequence. In one embodiment, the inhibitory molecule targets the NeST sequence of SEQ ID NO:1. In certain embodiments, the inhibitory RNA molecule may be a double-stranded, small interfering RNA or a short hairpin RNA molecule (shRNA) comprising a stem-loop structure.

In one embodiment, the invention includes a method of modulating an immune response in a subject, the method comprising administering a therapeutically effective amount of a NeST inhibitor to the subject, wherein the NeST inhibitor is administered in an amount sufficient to decrease production of IFN-γ by leukocytes (e.g., T cells, natural killer cells, myeloid cells, dendritic cells, and macrophages).

An “effective amount” of a NeST inhibitor (e.g., microRNA, siRNA, piRNA, snRNA, antisense oligonucleotide, ribozyme, or small molecule inhibitor) is an amount sufficient to effect beneficial or desired results, such as an amount that reduces NeST activity, for example, by interfering with transcription of NeST, binding of NeST to WDR5, or activation of IFN-γ gene expression. In some embodiments, a NeST inhibitor reduces the amount and/or activity of NeST by at least about 10% to about 100%, 20% to about 100%, 30% to about 100%, 40% to about 100%, 50% to about 100%, 60% to about 100%, 70% to about 100%, 10% to about 90%, 20% to about 85%, 40% to about 84%, 60% to about 90%, including any percent within these ranges, such as but not limited to 15%, 20%, 25%, 30%, 35%, 40%, 45%, 50%, 55%, 60%, 65%, 70%, 75%, 80%, 85%, 90%, 95%, 96%, 97%, 98%, and 99%. Preferably, the NeST inhibitor also reduces inflammation in a subject.

In certain embodiments, NeST or a NeST inhibitor (e.g., microRNA, siRNA, piRNA, snRNA, or antisense oligonucleotide) is expressed in vivo from a vector. A “vector” is a composition of matter which can be used to deliver a nucleic acid of interest to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, lentiviral vectors, and the like. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

In one embodiment, an expression vector for expressing NeST or an inhibitor of NeST comprises a promoter “operably linked” to a polynucleotide encoding NeST or an inhibitor of NeST. The phrase “operably linked” or “under transcriptional control” as used herein means that the promoter is in the correct location and orientation in relation to a polynucleotide to control the initiation of transcription by RNA polymerase and expression of the polynucleotide.

In certain embodiments, the nucleic acid encoding a polynucleotide of interest is under transcriptional control of a promoter. A “promoter” refers to a DNA sequence recognized by the synthetic machinery of the cell, or introduced synthetic machinery, required to initiate the specific transcription of a gene. The term promoter will be used here to refer to a group of transcriptional control modules that are clustered around the initiation site for RNA polymerase I, II, or III. Typical promoters for mammalian cell expression include the SV40 early promoter, a CMV promoter such as the CMV immediate early promoter (see, U.S. Pat. Nos. 5,168,062 and 5,385,839, incorporated herein by reference in their entireties), the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression. These and other promoters can be obtained from commercially available plasmids, using techniques well known in the art. See, e.g., Sambrook et al., supra. Enhancer elements may be used in association with the promoter to increase expression levels of the constructs. Examples include the SV40 early gene enhancer, as described in Dijkema et al., EMBO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence.

Typically, transcription terminator/polyadenylation signals will also be present in the expression construct. Examples of such sequences include, but are not limited to, those derived from SV40, as described in Sambrook et al., supra, as well as a bovine growth hormone terminator sequence (see, e.g., U.S. Pat. No. 5,122,458). Additionally, 5′-UTR sequences can be placed adjacent to the coding sequence in order to enhance expression of the same. Such sequences include UTRs which include an Internal Ribosome Entry Site (IRES) present in the leader sequences of picornaviruses such as the encephalomyocarditis virus (EMCV) UTR (Jang et al. J. Virol. (1989) 63:1651-1660. Other picornavirus UTR sequences that will also find use in the present invention include the polio leader sequence and hepatitis A virus leader and the hepatitis C IRES.

In certain embodiments of the invention, the cells containing nucleic acid constructs of the present invention may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Fluorescent markers (e.g., GFP, EGFP, Dronpa, mCherry, mOrange, mPlum, Venus, YPet, phycoerythrin), or immunologic markers can also be employed. The selectable marker employed is not believed to be important, so long as it is capable of being expressed simultaneously with the nucleic acid encoding a gene product. Further examples of selectable markers are well known to one of skill in the art.

There are a number of ways in which expression vectors may be introduced into cells. In certain embodiments of the invention, the expression construct comprises a virus or engineered construct derived from a viral genome. A number of viral based systems have been developed for gene transfer into mammalian cells. These include adenoviruses, retroviruses (γ-retroviruses αnd lentiviruses), poxviruses, adeno-associated viruses, baculoviruses, and herpes simplex viruses (see e.g., Warnock et al. (2011) Methods Mol. Biol. 737:1-25; Walther et al. (2000) Drugs 60(2):249-271; and Lundstrom (2003) Trends Biotechnol. 21(3):117-122; herein incorporated by reference in their entireties). The ability of certain viruses to enter cells via receptor-mediated endocytosis, to integrate into host cell genomes and express viral genes stably and efficiently have made them attractive candidates for the transfer of foreign genes into mammalian cells.

For example, retroviruses provide a convenient platform for gene delivery systems. Selected sequences can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; Boris-Lawrie and Temin (1993) Cur. Opin. Genet. Develop. 3:102-109; and Ferry et al. (2011) Curr. Pharm. Des. 17(24):2516-2527). Lentiviruses are a class of retroviruses that are particularly useful for delivering polynucleotides to mammalian cells because they are able to infect both dividing and nondividing cells (see e.g., Lois et al (2002) Science 295:868-872; Durand et al. (2011) Viruses 3(2):132-159; herein incorporated by reference).

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering the polynucleotides of the present invention is the enterically administered recombinant poxvirus vaccines described by Small, Jr., P. A., et al. (U.S. Pat. No. 5,676,950, issued Oct. 14, 1997, herein incorporated by reference).

Additional viral vectors which will find use for delivering the nucleic acid molecules of interest include those derived from the pox family of viruses, including vaccinia virus and avian poxvirus. By way of example, vaccinia virus recombinants expressing a nucleic acid molecule of interest (e.g., NeST or an inhibitor of NeST) can be constructed as follows. The DNA encoding the particular nucleic acid sequence is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the sequences of interest into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the nucleic acid molecules of interest. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the Alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides of the present invention. For a description of Sindbis-virus derived vectors useful for the practice of the instant methods, see, Dubensky et al. (1996) J. Virol. 70:508-519; and International Publication Nos. WO 95/07995, WO 96/17072; as well as, Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference. Particularly preferred are chimeric alphavirus vectors comprised of sequences derived from Sindbis virus and Venezuelan equine encephalitis virus. See, e.g., Perri et al. (2003) J. Virol. 77: 10394-10403 and International Publication Nos. WO 02/099035, WO 02/080982, WO 01/81609, and WO 00/61772; herein incorporated by reference in their entireties.

A vaccinia based infection/transfection system can be conveniently used to provide for inducible, transient expression of the polynucleotides of interest (e.g., NeST or an inhibitor of NeST) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide of interest, driven by a T7 promoter. The polymerase expressed in the cytoplasm from the vaccinia virus recombinant transcribes the transfected DNA into RNA. The method provides for high level, transient, cytoplasmic production of large quantities of RNA. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

As an alternative approach to infection with vaccinia or avipox virus recombinants, or to the delivery of nucleic acids using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more template. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present invention. These include the use of calcium phosphate precipitation, DEAE-dextran, electroporation, direct microinjection, DNA-loaded liposomes, lipofectamine-DNA complexes, cell sonication, gene bombardment using high velocity microprojectiles, and receptor-mediated transfection (see, e.g, Graham and Van Der Eb (1973) Virology 52:456-467; Chen and Okayama (1987) Mol. Cell. Biol. 7:2745-2752; Rippe et al. (1990) Mol. Cell. Biol. 10:689-695; Gopal (1985) Mol. Cell. Biol. 5:1188-1190; Tur-Kaspa et al. (1986) Mol. Cell. Biol. 6:716-718; Potter et al. (1984) Proc. Natl. Acad. Sci. USA 81:7161-7165); Harland and Weintraub (1985) J. Cell Biol. 101:1094-1099); Nicolau and Sene (1982) Biochim. Biophys. Acta 721:185-190; Fraley et al. (1979) Proc. Natl. Acad. Sci. USA 76:3348-3352; Fechheimer et al. (1987) Proc Natl. Acad. Sci. USA 84:8463-8467; Yang et al. (1990) Proc. Natl. Acad. Sci. USA 87:9568-9572; Wu and Wu (1987) J. Biol. Chem. 262:4429-4432; Wu and Wu (1988) Biochemistry 27:887-892; herein incorporated by reference). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (Proc. Natl. Acad. Sci. USA (1984) 81:7529-7533) successfully injected polyomavirus DNA in the form of calcium phosphate precipitates into liver and spleen of adult and newborn mice demonstrating active viral replication and acute infection. Benvenisty and Neshif (Proc. Natl. Acad. Sci. USA (1986) 83:9551-9555) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment, a naked DNA expression construct may be transferred into cells by particle bombardment. This method depends on the ability to accelerate DNA-coated microprojectiles to a high velocity allowing them to pierce cell membranes and enter cells without killing them (Klein et al. (1987) Nature 327:70-73). Several devices for accelerating small particles have been developed. One such device relies on a high voltage discharge to generate an electrical current, which in turn provides the motive force (Yang et al. (1990) Proc. Natl. Acad. Sci. USA 87:9568-9572). The microprojectiles may consist of biologically inert substances, such as tungsten or gold beads.

In a further embodiment, the expression construct may be delivered using liposomes. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat (1991) Liver Diseases, Targeted Diagnosis and Therapy Using Specific Receptors and Ligands, Wu et al. (Eds.), Marcel Dekker, NY, 87-104). Also contemplated is the use of lipofectamine-DNA complexes.

In certain embodiments of the invention, the liposome may be complexed with a hemagglutinating virus (HVJ). This has been shown to facilitate fusion with the cell membrane and promote cell entry of liposome-encapsulated DNA (Kaneda et al. (1989) Science 243:375-378). In other embodiments, the liposome may be complexed or employed in conjunction with nuclear non-histone chromosomal proteins (HMG-I) (Kato et al. (1991) J. Biol. Chem. 266(6):3361-3364). In yet further embodiments, the liposome may be complexed or employed in conjunction with both HVJ and HMG-I. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu (1993) Adv. Drug Delivery Rev. 12:159-167).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin (see, e.g., Wu and Wu (1987), supra; Wagner et al. (1990) Proc. Natl. Acad. Sci. USA 87(9):3410-3414). Recently, a synthetic neoglycoprotein, which recognizes the same receptor as ASOR, has been used as a gene delivery vehicle (Ferkol et al. (1993) FASEB J. 7:1081-1091; Perales et al. (1994) Proc. Natl. Acad. Sci. USA 91(9):4086-4090), and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (Methods Enzymol. (1987) 149:157-176) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In a particular example, an oligonucleotide may be administered in combination with a cationic lipid. Examples of cationic lipids include, but are not limited to, lipofectin, DOTMA, DOPE, and DOTAP. The publication of WO/0071096, which is specifically incorporated by reference, describes different formulations, such as a DOTAP:cholesterol or cholesterol derivative formulation that can effectively be used for gene therapy. Other disclosures also discuss different lipid or liposomal formulations including nanoparticles and methods of administration; these include, but are not limited to, U.S. Patent Publication 20030203865, 20020150626, 20030032615, and 20040048787, which are specifically incorporated by reference to the extent they disclose formulations and other related aspects of administration and delivery of nucleic acids. Methods used for forming particles are also disclosed in U.S. Pat. Nos. 5,844,107, 5,877,302, 6,008,336, 6,077,835, 5,972,901, 6,200,801, and 5,972,900, which are incorporated by reference for those aspects.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

The NeST or inhibitor of NeST may comprise a detectable label in order to determine cellular uptake efficiency, quantitate binding at target sites, or visualize localization. Detectable labels suitable for use in the present invention include any composition detectable by spectroscopic, photochemical, biochemical, immunochemical, electrical, optical, or chemical means. Useful labels in the present invention include biotin or other streptavidin-binding proteins for staining with labeled streptavidin conjugate, magnetic beads (e.g., Dynabeads), fluorescent dyes (e.g., phycoerythrin, YPet, fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, ³²P), enzymes (e.g., horseradish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. In addition, magnetic resonance imaging (MRI) contrast agents (e.g., gadodiamide, gadobenic acid, gadopentetic acid, gadoteridol, gadofosveset, gadoversetamide, gadoxetic acid), and computed tomography (CT) contrast agents (e.g., Diatrizoic acid, Metrizoic acid, Iodamide, Iotalamic acid, Ioxitalamic acid, Ioglicic acid, Acetrizoic acid, Iocarmic acid, Methiodal, Diodone, Metrizamide, Iohexyl, Ioxaglic acid, Iopamidol, Iopromide, Iotrolan, Ioversol, Iopentol, Iodixanol, Iomeprol, Iobitridol, Ioxilan, Iodoxamic acid, Iotroxic acid, Ioglycamic acid, Adipiodone, Iobenzamic acid, Iopanoic acid, Iocetamic acid, Sodium iopodate, Tyropanoic acid, Calcium iopodate) are useful as labels in medical imaging. Patents teaching the use of such labels include U.S. Pat. Nos. 3,817,837; 3,850,752; 3,939,350; 3,996,345; 4,277,437; 4,275,149; 4,366,241; 5,798,092; 5,695,739; 5,733,528; and 5,888,576.

B. Applications

The methods of the invention are useful for treating various disorders including, e.g., infectious diseases, such as caused by a virus or cellular pathogen; increasing an immune response to a cancerous cell or tumor in an individual; enhancing an immune response in an individual who is immunodeficient or immunocompromised; or for decreasing inflammation, such as caused by an autoimmune disease, a neurodegenerative disease, a cardiovascular disease, damaged tissue, or a wound.

Inflammatory conditions and autoimmune diseases that may be treated by the methods of the invention include, but are not limited to multiple sclerosis (MS), rheumatoid arthritis (RA), reactive arthritis, psoriasis, pemphigus vulgaris, Sjogren's disease, autoimmune thyroid disease (AITD), Hashimoto's thyroiditis, myasthenia gravis, insulin dependent diabetes mellitus (IDDM), stomatitis, lupus erythematosus, ischemic heart disease, atherosclerosis, cancer, fibrosis, inflammatory bowel disease, inflammatory myopathy, giant cell arteritis (GCA), asthma, allergy, Parkinson's disease, or Alzheimer's disease. Treatment of primates, more particularly humans is of interest, but other mammals may also benefit from treatment, particularly domestic animals such as equine, bovine, ovine, feline, canine, murine, lagomorpha, and the like.

In some embodiments, the methods of the invention can be used to increase or decrease an immune response for treating: (a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia), a picornavirus (e.g., rhinovirus or enterovirus), an orthomyxovirus (e.g., influenza virus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as human immunodeficiency virus (HIV)); (b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, and Bordetella; (c) other infectious diseases, such as, but not limited to Chlamydia infection, fungal diseases including but not limited to candidiasis, aspergillosis, blastomycosis, histoplasmosis, cryptococcal meningitis, and parasitic diseases including but not limited to malaria, Pneumocystis carinii pneumonia, leishmaniasis, cryptosporidiosis, toxoplasmosis, and trypanosome infection; and (d) neoplastic diseases, such as, for example, intraepithelial neoplasias, cervical dysplasia, actinic keratosis, basal cell carcinoma, squamous cell carcinoma, renal cell carcinoma, Kaposi's sarcoma, melanoma, renal cell carcinoma, leukemias including but not limited to myelogeous leukemia, chronic lymphocytic leukemia, multiple myeloma, non-Hodgkin's lymphoma, cutaneous T-cell lymphoma, B-cell lymphoma, and hairy cell leukemia, and other cancers.

In addition, NeST may be administered in combination with a vaccine to augment the immune response to a cellular pathogen or cancerous cells. NeST may also be used to enhance the immune response to antibiotic-resistant bacteria and for treating sepsis or food poisoning.

C. Pharmaceutical Compositions and Administration

The present invention also encompasses pharmaceutical compositions comprising NeST or one or more NeST inhibitors and a pharmaceutically acceptable carrier. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

Colloidal dispersion systems, such as macromolecule complexes, nanocapsules, microspheres, beads, and lipid-based systems including oil-in-water emulsions, micelles, mixed micelles, and liposomes, may be used as delivery vehicles for NeST or inhibitors of NeST described herein. Commercially available fat emulsions that are suitable for delivering the nucleic acids to tissues include Intralipid, Liposyn, Liposyn II, Liposyn III, Nutrilipid, and other similar lipid emulsions. A preferred colloidal system for use as a delivery vehicle in vivo is a liposome (i.e., an artificial membrane vesicle). The preparation and use of such systems is well known in the art. Exemplary formulations are also disclosed in U.S. Pat. No. 5,981,505; U.S. Pat. No. 6,217,900; U.S. Pat. No. 6,383,512; U.S. Pat. No. 5,783,565; U.S. Pat. No. 7,202,227; U.S. Pat. No. 6,379,965; U.S. Pat. No. 6,127,170; U.S. Pat. No. 5,837,533; U.S. Pat. No. 6,747,014; and WO 03/093449, which are herein incorporated by reference in their entireties.

One will generally desire to employ appropriate salts and buffers to render delivery vehicles stable and allow for uptake by target cells. Buffers also will be employed when recombinant cells are introduced into a patient. Aqueous compositions of the present invention comprise an effective amount of the delivery vehicle, dissolved or dispersed in a pharmaceutically acceptable carrier or aqueous medium. The phrases “pharmaceutically acceptable” or “pharmacologically acceptable” refers to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the nucleic acids of the compositions.

Compositions for use in the invention will comprise a therapeutically effective amount of NeST or at least one NeST inhibitor. For example, an “effective amount” of NeST is an amount sufficient to increase production of IFN-γ from leukocytes. By “therapeutically effective dose or amount” of NeST is intended an amount that, when administered as described herein, brings about a positive therapeutic response, such as improved recovery from an infectious disease or cancer. Improved recovery may involve activation of the immune system against pathogens or cancerous cells by raising the level of IFN-γ produced by leukocytes to provide immunoregulatory, antiviral, antiseptic, anti-tumor, or anti-metastatic activity. An “effective amount” of a NeST inhibitor (e.g., microRNA, siRNA, piRNA, snRNA, antisense nucleic acid, ribozyme, or small molecule inhibitor) is an amount sufficient to inhibit the activity of NeST, for example by interfering with transcription of NeST, binding of NeST to WDR5, or activation of IFN-γ gene expression. By “therapeutically effective dose or amount” of a NeST inhibitor is intended an amount that, when administered as described herein, brings about a positive therapeutic response, such as improved recovery from an inflammatory condition or autoimmune disorder. Improved recovery may include reducing inflammation associated with a disease such as, but not limited to an autoimmune disease, a cardiovascular disease, or a neurodegenerative disorder, or resulting from the inflammatory response to damaged cells or wounds. An effective amount of NeST or a NeST inhibitor can be administered in one or more administrations, applications or dosages.

The pharmaceutical preparation can be in the form of a liquid solution or suspension immediately prior to administration, but may also take another form such as a syrup, cream, ointment, tablet, capsule, powder, gel, matrix, suppository, or the like. The pharmaceutical compositions comprising NeST or one or more NeST inhibitors may be administered in accordance with any medically acceptable method known in the art. Suitable routes of administration include parenteral administration, such as subcutaneous (SC), intraperitoneal (IP), intramuscular (IM), intravenous (IV), or infusion, or oral, pulmonary, nasal, topical, transdermal, and so forth. In some embodiments of the invention, the pharmaceutical composition comprising the NeST or NeST inhibitor is administered by IM or SC injection, particularly by IM or SC injection locally, for example, to an infected, cancerous, or inflamed region needing treatment.

In another embodiment, the pharmaceutical compositions comprising NeST or one or more NeST inhibitors are administered prophylactically, e.g., to prevent inflammation, infection, or tumor growth. Such prophylactic uses will be of particular value for subjects with a disease or who have a genetic predisposition to developing infections (e.g., chronic granulomatous disease, osteopetrosis), immunodeficiency, or inflammation, or who are immunocompromised.

The actual dose to be administered will vary depending upon the mode of administration, the frequency of administration (i.e., daily, or intermittent administration, such as twice- or thrice-weekly), the particular disease undergoing therapy, the severity of the disease, the history of the disease, whether the individual is undergoing concurrent therapy with another therapeutic agent, and the age, height, weight, health, and physical condition of the individual undergoing therapy. Generally, a higher dosage of an agent is preferred with increasing weight of the subject undergoing therapy.

Therapeutically effective amounts can be determined by those skilled in the art, and will be adjusted to the particular requirements of each particular case. Generally, a therapeutically effective amount will range from about 0.50 mg to 5 grams daily, more preferably from about 5 mg to 2 grams daily, even more preferably from about 7 mg to 1.5 grams daily. Preferably, such doses are in the range of 10-600 mg four times a day (QID), 200-500 mg QID, 25-600 mg three times a day (TID), 25-50 mg TID, 50-100 mg TID, 50-200 mg TID, 300-600 mg TID, 200-400 mg TID, 200-600 mg TID, 100 to 700 mg twice daily (BID), 100-600 mg BID, 200-500 mg BID, or 200-300 mg BID. An appropriate effective amount can be readily determined by one of skill in the art. A “therapeutically effective amount” will fall in a relatively broad range that can be determined through routine trials using in vitro and in vivo models known in the art.

In certain embodiments, multiple therapeutically effective doses of NeST or at least one NeST inhibitor will be administered according to a daily dosing regimen, or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, and so forth. For example, in some embodiments, NeST or at least one NeST inhibitor will be administered twice-weekly or thrice-weekly for an extended period of time, such as for 1, 2, 3, 4, 5, 6, 7, 8 . . . 10 . . . 15 . . . 24 weeks, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy (i.e., twice-weekly or thrice-weekly administration of a therapeutically effective dose) for one or more weekly cycles until the desired therapeutic response is achieved.

The pharmaceutical forms suitable for injectable use or catheter delivery include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present invention generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like).

Once formulated, the compositions of the invention can be administered directly to the subject (e.g., as described above) or, alternatively, delivered ex vivo, to cells (e.g., leukocytes) derived from the subject, using methods such as those described above. For example, methods for the ex vivo delivery and reimplantation of transformed cells into a subject are known in the art and can include, e.g., dextran-mediated transfection, calcium phosphate precipitation, polybrene mediated transfection, lipofectamine and LT-1 mediated transfection, protoplast fusion, electroporation, encapsulation of the polynucleotide(s) in liposomes, and direct microinjection of the DNA into nuclei.

D. Kits

Any of the compositions described herein may be included in a kit. For example, NeST or at least one NeST inhibitor may be included in a kit. The kit may also include one or more transfection reagents to facilitate delivery of polynucleotides to cells.

The components of the kit may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

Such kits may also include components that preserve or maintain the NeST or NeST inhibitors or that protect against their degradation. Such components may be RNAse-free or protect against RNAses. Such kits generally will comprise, in suitable means, distinct containers for each individual reagent or solution.

A kit will also include instructions for employing the kit components as well the use of any other reagent not included in the kit. Instructions may include variations that can be implemented. A kit may also include utensils or devices for administering the miRNA agonist or inhibitor by various administration routes, such as parenteral or catheter administration or coated stent.

E. Screening Methods

Also provided is a screening assay to identify an agent that modulates IFN-γ production in vivo. In particular embodiments, the method may be employed to identify an agent can be used to modulate the immune system or treat a mammal for an infectious disease. In exemplary embodiments, the method may comprise: a) identifying an agent that modulates NeST expression in a leukocyte; and b) testing said agent in vivo to determine whether it can decrease the severity of at least one symptom of inflammation, or treat an animal for an infection. The agent can be, for example, an inhibitory RNA, a NeST cDNA, or a small molecule. Inhibitory RNA, DNA and hybrid oligonucleotides are set forth above.

The term “agent” as used herein describes any molecule, e.g. protein or non-protein organic or inorganic pharmaceutical. Agents of particular interest are those that increase or decrease NeST expression. A plurality of assays may be run in parallel with different agent concentrations to obtain a differential response to the various concentrations. One of these concentrations may serve as a negative control, i.e. at zero concentration or below the level of detection.

The terms “candidate agent”, “test agent”, “agent”, “substance” and “compound” are used interchangeably herein. Candidate agents encompass numerous chemical classes, typically synthetic, semi-synthetic, or naturally-occurring inorganic or organic molecules. Candidate agents include those found in large libraries of synthetic or natural compounds. For example, synthetic compound libraries are commercially available from Maybridge Chemical Co. (Trevillet, Cornwall, UK), ComGenex (South San Francisco, Calif.), and MicroSource (New Milford, Conn.). Alternatively, libraries of natural compounds in the form of bacterial, fungal, plant and animal extracts are available from Pan Labs (Bothell, Wash.) or are readily producible.

Candidate agents may be small organic or inorganic compounds having a molecular weight of more than 50 and less than about 2,500 Da. Candidate agents may comprise functional groups necessary for structural interaction with proteins, particularly hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. New potential therapeutic agents may also be created using methods such as rational drug design or computer modeling.

Screening may be directed to known pharmacologically active compounds and chemical analogs thereof, or to new agents with unknown properties such as those created through rational drug design.

Agents that modulate a phenotype may decrease NeST expression by at least 10%, at least 20%, at least 30%, at least 40%, at least 50%, at least 60%, at least 70%, at least 80%, or at least 90%, or more, relative to a control that has not been exposed to the agent. In alternative cases, agents that modulate a phenotype may increase NeST expression by at least 10%, at least 30%, at least 50%, at least 60%, at least 100%, at least 200%, or at least 500%, or more, relative to a control that has not been exposed to the agent.

Agents that modulate the NeST expression may be subjected to directed or random and/or directed chemical modifications, such as acylation, alkylation, esterification, amidification, etc. to produce structural analogs. Such structural analogs include those that increase bioavailability, and/or reduced cytotoxicity. Those skilled in the art can readily envision and generate a wide variety of structural analogs, and test them for desired properties such as increased bioavailability and/or reduced cytotoxicity, etc.

NeST expression can be measured using any suitable method for assaying RNA expression. The effect of a candidate agent may be determined by measuring the RNA at several time points. For example, the production of RNA may be measured 5 minutes, 30 minutes, 1 hour, 2 hours, 4 hours, 8 hours, 12 hours, 24 hours, 36 hours, 48 hours, 72 hours, 120 hours, 1 week, 2 week, and up to 1 month, after contacting a cell with a candidate agent.

After identifying an agent that modulates NeST RNA production, the method may comprise testing the agent in vivo to determine whether it can decrease the severity of at least one symptom of inflammation, or treat an animal for a pathogen infection. Any phenotype produced in the in vivo system be monitored at different points before and after administering the candidate agent to the animal. For example, the effect of a candidate agent may be determined by measuring a phenotype at several time points. For example, the production of phenotype may be measured at time 0 hours, 12 hours, 24 hours, 36 hours, 48 hours, 72 hours, 120 hours, 1 week, 2 week, 1 month, 2 months, 3 months, 5 months, etc., after contacting the cell with a candidate agent.

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### The NeST Long Noncoding RNA Controls Microbial Susceptibility and Epigenetic Activation of the Interferon-γ Locus

### Introduction

Long noncoding RNAs (lncRNAs) are increasingly appreciated as regulators of cell-specific gene expression. Here, an enhancer-like lncRNA, referred to as NeST (nettoie Salmonella pas Theiler's [cleanup Salmonella not Theiler's]), is shown to be causal for all phenotypes conferred by murine viral susceptibility locus Tmevp3. This locus was defined by crosses between SJL/J and B10.S mice and contains several candidate genes, including NeST. The SJL/J-derived locus confers higher lncRNA expression, increased interferon-gamma (IFN-γ) abundance in activated CD8⁺ T cells, increased Theiler's virus persistence, and decreased Salmonella enterica pathogenesis. Transgenic expression of NeST lncRNA alone was sufficient to confer all phenotypes of the SJL/J locus. NeST RNA was found to bind WDR5, a component of the histone H3 lysine 4 methyltransferase complex, and to alter histone 3 methylation at the IFN-γ locus. Thus, this lncRNA regulates epigenetic marking of IFN-γ-encoding chromatin, expression of IFN-γ, and susceptibility to a viral and a bacterial pathogen.

Theiler's virus, a picornavirus, is a natural pathogen of mice. The ability of inbred mice to clear Theiler's infection varies greatly from strain to strain, and, because the phenotype can be conferred by bone marrow transfer (Aubagnac et al. (2002) J. Virol. 76:5807-5812; Brahic et al. (2005) Annu. Rev. Microbiol. 59:279-298; Vigneau et al. (2003) J. Virol. 77:5632-5638), is likely to result from different immune responses to the pathogen. A major effect is conferred by the H2 locus. Two additional loci that affect Theiler's virus clearance were mapped by crosses between HT-bearing SJL/J and B10.S mice. Whereas B10.S mice can clear the virus, SJL/J mice become persistently infected and develop demyelinating lesions similar to those observed in human multiple sclerosis (Aubagnac et al. (2002) J. Virol. 76:5807-5812; Bureau et al. (1993) Nat. Genet. 5:87-91).

One of these loci, Tmevp3 (Theiler's murine encephalitis virus persistence 3; FIG. 1B), was mapped to a 550 kb interval on murine chromosome 10 (Levillayer et al. (2007) Genetics 176:1835-1844). Congenic mouse lines were developed by crossing SJL/J to B10.S and back-crossing to each parental line for 10 to 12 generations (Bihl et al. (1999) Genetics 152:385-392; Bureau et al. (1993) Nat. Genet. 5:87-91; Levillayer et al., supra). The B10.S.Tmevp3^{SJL/J} line is congenic with B10.S but contains the Tmevp3 locus from SJL/J and is unable to clear persistent infections. Conversely, the SJL/J.Tmevp3^{B10.S} line is congenic with SJL/J but contains the Tmevp3 locus from B10.S and successfully clears infections. Analysis of SNPs in the smallest introgressed B10.S-derived region revealed a small number of polymorphic genes, including those that encode Mdm1 (Chang et al. (2008) Hum. Mol. Genet. 17:3929-3941), the potent immune cytokines IL-22 and IFN-γ, and the lncRNA (FIG. 1C).

Here, we show additional phenotypes associated with the Tmevp3 locus. In addition to the failure to clear Theiler's virus, the SJL/J-derived alleles also confer both resistance to lethal infection with Salmonella enterica Typhimurium and inducible synthesis of IFN-γ in CD8⁺ T cells. We show that NeST lncRNA is sufficient to confer these disparate phenotypes, demonstrating its crucial role in the host response to pathogens and illustrating an integral function for lncRNAs in immune regulation and susceptibility to infectious disease.

Experimental Procedures

Ethics Statement

All animal experiments were carried out at the Stanford RAF under the supervision of the Veterinary Service Center at the Department of Comparative Medicine at Stanford, which is accredited by the Association for Assessment and Accreditation of Laboratory Animal Care (AAALAC). All experiments were approved by the Administrative Panel of Laboratory Animal Care (APLAC) and are consistent with federal, state and local guidelines for laboratory animal care.

Viral and Bacterial Strains and Culture

The DA strain of Theiler's virus was produced by transfecting the PTM762 plasmid into BHK-21 cells as previously described (McAllister et al. (1989) Microb. Pathog. 7:381-388; herein incorporated by reference). Salmonella enterica serovar Typhimurium SL1344 strain was used (Subbaiah and Stocker (1964) Nature 201:1298-1299; herein incorporated by reference).

Mouse Strains and Transgenic Line Design

Congenic B10.S Tmevp3^{SJL/J} and SJL/J.Tmevp3^{B10.S} mice were imported from the Pasteur Institute animal facility and colonies were established. B10.S and SJL/J mice were obtained from the Jackson Laboratory (Bar Harbor, Me.). (Balb/c×129) F1 pseudopregnant female mice were provided by Drs. Hugh McDevitt and Grete Sonderstrup (Stanford, Calif.). All mice were bred at the Stanford Research Animal Facility (RAF) except for SJLJ/J mice, which were purchased at 4 weeks old and housed in the Stanford RAF for at least 6 weeks prior to all experiments.

B10.S mice that express NeST RNA transgenically were developed by pronuclear microinjections. NeST cDNA was cloned into the unique Sail site of the p428 expression vector, placing it downstream of a CD4⁺ and CD8⁺ T cell-specific promoter and upstream of an SV40 polyadenylation signal. The p428 plasmid contains a mouse Cd4 promoter with a 428 bp silencer deletion, which allows specific expression in both CD4⁺ and CD8⁺ T cells (Sawada et al. (1994) Cell 77:917-929; herein incorporated by reference). The T cell promoter-NeST transgene was released from the vector backbone using Not I. The transgene was gel purified and injected into fertilized B10.S oocytes obtained via natural estrous cycle. Estrous cycle determination, recovery of single cell embryos, microinjection procedures and transfer to pseudopregnant females has been previously described (Singer et al. (1998) Diabetes 47:1570-1577). Transgenic founders were identified by quantitative PCR (qPCR) (Transnetyx, Tenn.) and mated to B10.S wild-type mice. Approximate copy numbers of integrated transgenes were calculated by qPCR analysis of over 100 mice. All experiments included littermate controls.

Inoculations

Four or five week old mice were inoculated with Theiler's virus as previously described (Bureau et al. (1992) J. Viol. 66:4698-4704) with the following modifications. Prior to intracerebral infections, mice were anesthetized with 2,2,2-tribromoethanol/2-methyl-2-butanol (Sigma-Aldrich, St. Louis, Mo.). A solution that contained 125 mg of this compound dissolved in 0.25 ml of 2-methyl-2-butanol was added to 10 ml of sterile distilled water. The solution was filter sterilized and kept at 4′C in the dark for a maximum of 10 days. Each mouse received 20 ml/gram of anesthetic by intraperitoneal injection. Mice were inoculated with Theiler's virus by injecting 40 ml of viral suspension in the left hemisphere. Mice were dissected at the indicated day after inoculation. To avoid contamination with peripheral blood, mice were perfused with PBS prior to brain and spinal cord dissections. Homogenized tissues were assayed for viral titers by plaque assay.

Mice inoculated with Salmonella enterica Typhimurium were 10-12 weeks old; the lethality of bacterial infection was found to be very sensitive to animal age. All experiments reported here include concurrent controls. For oral Salmonella infections, mice were denied food for 12 hours and then provided with bread containing the indicated bacterial inoculum (Broz et al. (2010) J. Exp. Med. 207:1745-1755). For intraperitoneal inoculations, mice were injected with live bacteria in 100 ml of PBS. Bacteria were grown for 10 hours aerobically at 37′C. Colony-forming units (CFU) were determined by plating after inoculation. Tissues were collected at the indicated day after inoculation, weighed and homogenized in PBS. Dilutions were plated on LB plates supplemented with 100 mg/ml of streptomycin to determine CFUs. Mice used for lipopolysaccharide (LPS) injections were 10-12 weeks old. 100 mg of LPS (Sigma-Aldrich, St. Louis, Mo.) was delivered by intraperitoneal injection in 100 ml of sterile PBS. Mice were monitored for mortality.

Cell Culture, Infection, and Stimulation

Macrophage culture and infection were performed as previously described (Arpaia et al. (2011) Cell 144:675-688; Martinat et al. (2002) J. Viol. 76:12823-12833; herein incorporated by reference). Briefly, tibia and femur were dissected and bone marrow was flushed with 10 ml of DMEM (Invitrogen). The recovered cells were cultured in DMEM supplemented with 10% (v/v) FBS (Omega Scientific) and 10% (v/v) of mouse L-cell conditioned medium as a source of macrophage colony stimulating factor at a density of 5×10⁶ cells per plate. Three days after culturing, 4 ml of additional medium was added to each plate. On day six, cultured medium was removed and cells were incubated in PBS (without calcium and magnesium) for 20 minutes at 4′C. Macrophages were then detached from the plate by scraping.

For infections, macrophages were seeded at 2×10⁵ cells per well and infected at an MOI of 5 CFU/cell with bacteria in DMEM. Infection was carried out at 37′C for 30 minutes. After infection, cells were washed thoroughly with DMEM containing 100 mg/ml gentamicin and cultured in the presence of the antibiotic. Cells were harvested at 2, 4, 6, 8, and 24 hours after infection to measure intracellular bacteria.

For T cell culture ex vivo, splenocytes were prepared and CD3 T cell, CD4 T cell, or CD8 T were isolated with the use of kits from Miltenyi Biotec (Auburn, Calif.). Spleens were dissected and passed through a 70-micron cell strainer (BD Falcon, Franklin Lakes, N.J.) to dissociate splenocytes. Red blood cells were lysed in Gey solution (Sigma-Aldrich, St. Louis, Mo.). Remaining splenocytes were magnetically labeled using either CD3⁺ T cell, CD4⁺ T cell or CD8⁺ T cell Isolation Kits (Miltenyi Biotec, Auburn, Calif.). Cells were passed through a magnetic column and unretained T cells were collected. Nuclei were enriched as previously described (Huarte et al. (2010) Cell 142:409-419).

For T cell stimulation assays, cells were cultured in RPMI 1640 (Invitrogen) supplemented with 10% (v/v) fetal bovine serum (Omega Scientific), 50 mM β-mercaptoethanol and 1% (v/v) Pen Strep (Invitrogen). Cells were cultured for 10 hours prior to stimulation with 50 ng/ml phorbol 12-myristate 13-acetate and 1.5 mM ionomycin (Sigma-Aldrich). Cell pellets and supernatant were harvested at indicated times post stimulation and stored at −80° C.

WDR5 and Chromatin IP

NeST^{B10.S}, NeST^{SJL/J}, HOTTIP, HOTAIR, and U1-encoding cDNAs were cloned into a eukaryotic gene expression plasmid and cotransfected with pcDNA3.1 that did or did not express FlagWDR5.

Cells were lysed and immunoprecipitated as previously described, with modifications (Wang et al. (2011)) Nature 472:120-124; herein incorporated by reference). Chromatin IP (ChIP) and qPCR were carried out according to the Farnham protocol (O'Geen et al. (2011) Methods Mol. Biol. 791:265-286; herein incorporated by reference). Communoprecipitated RNA was extracted using TRIzol LS (Invitrogen) and RNeasy Mini Kits (QIAGEN), treated with TurboDNAFree (Ambion), and analyzed by SYBR Green Brilliant II qRT-PCR (Agilent). Controls that lacked reverse transcriptase demonstrated that no contaminating DNA was present (data not shown).

WDR5RIP

NeST^{B10.S} and NeST^{SJL/J} were cloned into KpnI- and XhoI-digested pcDNA3.1+ for eukaryotic gene expression. HEK293T cells (ATCC) were cotransfected using Lipofectamine 2000 (Invitrogen) with pcDNA3.1+FlagWDR5 and RNA expression plasmid (pcDNA3.1+HOTTIP, pcDNA3.1+NeST^{B10.S}, or pcDNA3.1+NeST^{SJL/J}). After 48-72 hours, cells were harvested by scraping into cold PBS, spun down and snap frozen in liquid nitrogen.

RNA and Cytokine Quantitation

Protein quantitation was performed using commercially available ELISA kits (R&D Systems, Inc., Minneapolis Minn.) or Luminex (Affymetrix, Inc, Santa Clara Calif.) according to manufacturer's instructions. For quantitative RT-PCR, total RNA from cells or tissue of interest was extracted with TRIzol Reagent (Invitrogen) according to the manufacturer's specifications and stored at −80° C. Standard curves were prepared using serial dilutions of known quantities of RNA. A plasmid that encodes NeST RNA from C57BL/6 mice was purchased from Invitrogen (IMAGE clone 599035). The three B10.S and 19 SJL/J polymorphisms (Table 1) were introduced by site-directed mutagenesis. Nonpolymorphic fragments of Ifng, Il-22, and Actin cDNAs were obtained from B10.S and SJL/J splenic RNA by reverse transcription and PCR; the PCR products were cloned using TOPO TA (Invitrogen) according to the manufacturer's instruction. Viral RNA was obtained by in vitro transcription of the PTMDA plasmid. Quantitative measurements of RNA prepared from cells were done by real time RT-PCR using the 7300 Time PCR System (Applied Biosystems, Carlsbad Calif.) and QuantiTect Sybr Green RT-PCR (QIAGEN cat. #204243). The primers for quantitation were:

(SEQ ID NO: 2)

| | |
|---|---|
| HOTTIP-F | 5′-CAAACTCCGTCCTCCAAAAC-3′, |

(SEQ ID NO: 3)

| | |
|---|---|
| HOTTIP-R | 5′-CAGTGAAGAGCGATCAGTGG-3′, |

(SEQ ID NO: 4)

| | |
|---|---|
| U1-F | 5′-ATACTTACCTGGCAGGGGAG-3′, |

(SEQ ID NO: 5)

| | |
|---|---|
| U1-R | 5′-CAGGGGGAAAGCGCGAACGCA-3′, |

(SEQ ID NO: 6)

| | |
|---|---|
| GAPDH-F | 5′-AGGTGGAGGAGTGGGTGTCGCTGTT-3′, |

(SEQ ID NO: 7)

| | |
|---|---|
| GAPDH-R | 5′-CCGGGAAACTGTGGCGTGATGG-3′, |

(SEQ ID NO: 8)

| | |
|---|---|
| NeST-F: | 5′-CAACGTACGCTGCCTCCCGATG-3′, |

(SEQ ID NO: 9)

| | |
|---|---|
| NeST-R: | 5′-CTATTTGGTCGAGTCTGACAGAG-3′, |

(SEQ ID NO: 10)

| | |
|---|---|
| Ifng-F: | 5′-CCTGTTACTACCTGACA CATTC-3′, |

(SEQ ID NO: 11)

| | |
|---|---|
| Ifng-R: | 5′-CCTTTACTTCACTGACCAATAAG-3′, |

(SEQ ID NO: 12)

| | |
|---|---|
| I1-22-F: | 5′-AGAACGTCTTCCAGGGTGAA-3′, |

(SEQ ID NO: 13)

| | |
|---|---|
| I1-22-R: | 5′-GCTACCTGA TGAAAGCAGG-3′, |

(SEQ ID NO: 14)

| | |
|---|---|
| Actin-F: | 5′-GCCTCGTCACCCACATAGGA-3′, |

(SEQ ID NO: 15)

| | |
|---|---|
| Actin-R: | 5′-AGGTGTGATGGTGGGAATGG-3′, |

(SEQ ID NO: 16)

| | |
|---|---|
| TMEV-F: | 5′-CCC AGTCCTCAGGAAATGAAGG, |
| and | |

(SEQ ID NO: 17)

| | |
|---|---|
| TMEV-R: | 5′-TCCAAAAGGAGAGGTGCCATAG |

(Jin et al. (2007) J. Virol. 81: 11690-11702).

TABLE 1
Tmevp3 Polymorphisms

| SNP Position | | | |
|---|---|---|---|
| (NCBI build 37) | SJL/J allele | B10.S allele | Gene |
| 117445288 | T | C | |
| 117445390 | C | A | |
| 117447530 | T | C | |
| 117454701 | T | C | |
| 117483642 | C | T | |
| 117489646 | A | C | |
| 117501980 | T | C | |
| 117558239 | T | A | |
| 117559089 | C | T | |
| 117562543 | G | A | |
| 117566343 | G | A | |
| 117566975 | C | T | |
| 117569076 | G | A | |
| 117569103 | C | T | |
| 117577506 | G | A | |
| 117577572 | T | C | |
| 117585333 | T | C | Mdm1 |
| 117596890 | C | T | Mdm1 |
| 117596918 | T | C | Mdm1 |
| 117597805 | A | G | Mdm1 |
| 117599623 | G | T | Mdm1 |
| 117599679 | A | G | Mdm1 |
| 117628776 | G | C | |
| 117641958 | A | | Il22 |
| 117641974 | C | G | Il22 |
| 117642001 | A | C | Il22 |
| 117642219 | G | A | Il22 |
| 117642263 | G | A | Il22 |
| 117642652 | C | T | Il22 |
| 117646528 | A | G | Il22 |
| 117646592 | T | A | Il22 |
| 117646691 | C | T | Il22 |
| 117646695 | C | T | Il22 |
| 117646895 | C | T | Il22 |
| 117649161 | A | G | |
| 117649176 | C | T | |
| 117724214 | C | T | |
| 117725266 | G | C | |
| 117731633 | T | C | Ilifb |
| 117740025 | T | G | |
| 117743071 | A | T | |
| 117744932 | T | C | |
| 117806632 | C | G | |
| 117834231 | C | T | |
| 117839439 | C | T | |
| 117859110 | C | T | |
| 117878011 | | T | Ifng |
| 117878012 | | T | Ifng |
| 117878013 | | T | Ifng |
| 117878014 | | T | Ifng |
| 117878015 | | T | Ifng |
| 117878016 | | T | Ifng |
| 117878017 | | T | Ifng |
| 117878018 | | T | Ifng |
| 117878019 | | T | Ifng |
| 117878020 | | T | Ifng |
| 117878021 | | T | Ifng |
| 117878022 | | C | Ifng |
| 117878023 | | C | Ifng |
| 117878024 | | T | Ifng |
| 117878025 | | T | Ifng |
| 117878026 | | T | Ifng |
| 117878057 | G | C | Ifng |
| 117878189 | G | C | Ifng |
| 117882274 | C | T | Ifng |
| 117882676 | G | A | Ifng |
| 117882750 | T | | Ifng |
| 117882750 | T | | Ifng |
| 117882750 | T | | Ifng |
| 117882772 | G | C | Ifng |
| 117888667 | A | G | |
| 117888719 | G | A | |
| 117890619 | G | C | |
| 117890823 | T | C | |
| 117898235 | C | A | |
| 117905878 | C | T | |
| 117939208 | G | A | NeST |
| 117939210 | T | A | NeST |
| 117939308 | C | T | NeST |
| 117939318 | G | A | NeST |
| 117939659 | T | G | NeST |
| 117939701 | C | G | NeST |
| 117939725 | A | C | NeST |
| 117939735 | A | G | NeST |
| 117944907 | C | T | NeST |
| 117948543 | C | T | NeST |
| 117948545 | C | A | NeST |
| 117948566 | A | G | NeST |
| 117948572 | C | T | NeST |
| 117948581 | G | A | NeST |
| 117948624 | G | A | NeST |
| 117951656 | T | C | NeST |
| 117951670 | C | T | NeST |
| 117956343 | C | T | NeST |
| 117985925 | G | A | NeST |
| 117993612 | A | T | NeST |
| 117993780 | T | C | NeST |
| 117993788 | C | T | NeST |

qPCR of Genome Segments following ChIP

ChIP and quantitative PCR was carried out following the Farnham protocol (O'Geen et al., supra). Briefly, approximately 5×10⁶ CD8⁺ T cells were purified and cross-linked with 1% formaldehyde. Chromatin was isolated using Nuclear Lysis Buffer (50 mM Tris-Cl pH 8.0, 10 mM EDTA, 1% SDS, PMSF, PI) and sonicated to reduce the size to approximately 1000 base pairs per fragment. Chromatin was incubated with 2 μg of H3K4me3 antibody (Abcam #ab8580) overnight at 4° C. Staph A cells were preblocked by incubation with 10 μl of 10 mg/ml BSA, then added for 15 minutes at room temperature. The Staph A cells were washed three times with dialysis buffer (2 mM EDTA, 50 mM Tris-Cl pH 8.0, 0.2% Sarkosyl, PMSF) and twice with washing buffer (100 mM Tris-Cl pH 9.0, 500 mM LiCl, 1% Igepal, 1% Deoxycholic Acid, PMSF). Material was eluted from precipitates and input controls using elution buffer (50 mM Tris-Cl pH 8.0, 10 mM EDTA, 1% SDS, PMSF) and vortexing at room temperature for 30 minutes. Crosslinking was released by overnight incubation at 67′C followed by RNase A treatment at 37′C for 30 minutes. The isolated DNA was purified using QIAGEN columns. For qPCR analysis of the eluted DNA was performed using Roche's Lightcycler. The primers for quantitation were:

| (SEQ ID NO: 18) | |
|---|---|
| Ifng1-F | 5′-CCATCGGCTGACCTAGAGAA-3′; |
| (SEQ ID NO: 19) | |
| Ifng1-R | 5′-ATGAGGAAGAGCTGCAAAGC-3′, |
| (SEQ ID NO: 20) | |
| Ifng2-F | 5′-ACCAAAACTACG CAGGGAAA-3′, |
| (SEQ ID NO: 21) | |
| Ifng2-R | 5′-GCTGGCTTTGATTCGATTGT-3′, |
| (SEQ ID NO: 22) | |
| Ifng3-F | 5′-TCAGAGGCCTGGACCATAAG-3′, |
| (SEQ ID NO: 23) | |
| Ifng3-R | 5′-GAAACTGCA AGGCCACAAAT-3′, |
| (SEQ ID NO: 24) | |
| Ifng4-F | 5′-ATTTGTGGCCTTGCAGTTTC-3′; |
| and | |
| (SEQ ID NO: 25) | |
| Ifng4-R | 5′-GGGCCCTTCCACTTACTTCT-3′. |

Statistical Analysis

Mean values and significance were determined using Student's t test. Survival curves were analyzed with the log rank test. The null hypothesis (that the strains compared were not different) was rejected when p values were <0.05. Instances when the observed differences could be reported with a confidence of 95% (*), 99% (**), or 99.9% (***) are denoted.

Results

Mapping the Tmevp3 Locus of Mouse Chromosome 10

To refine the borders of the Tmevp3 locus, we utilized the JAX mouse diversity genotyping array, which employs 623,124 SNPs and 916,269 invariant genomic probes. We also sequenced complementary DNAs (cDNAs) encoding interleukin-22 (IL-22), IFN-γ, and NeST RNA from SJL/J and B10.S mice, and added these findings to microarray results from the Jackson Laboratory (Bar Harbor, Me.; FIG. 1C) and the list of known polymorphisms in the locus (Table 1). Our results corroborated the presence of a unique introgressed region that contained the previously mapped Tmevp3 locus, and allowed us to refine its boundaries. The maximum sizes of the introgressed regions were 16×10⁶ bp and 550×10³ bp, respectively, for the B10.S.Tmevp3^{SJL/J} and SJL/J.Tmevp3B10.S congenic lines (FIG. 1C).

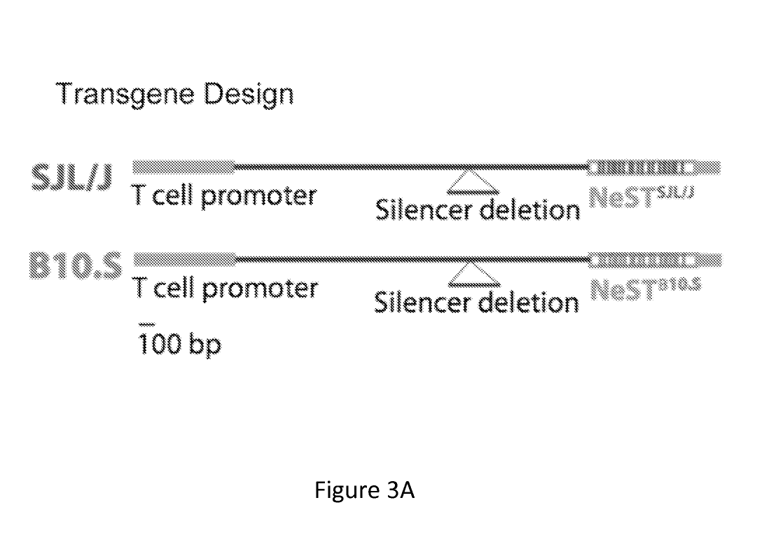

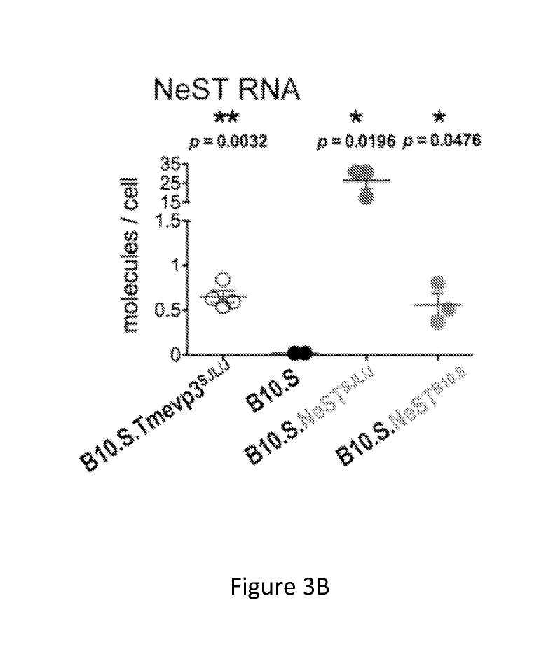

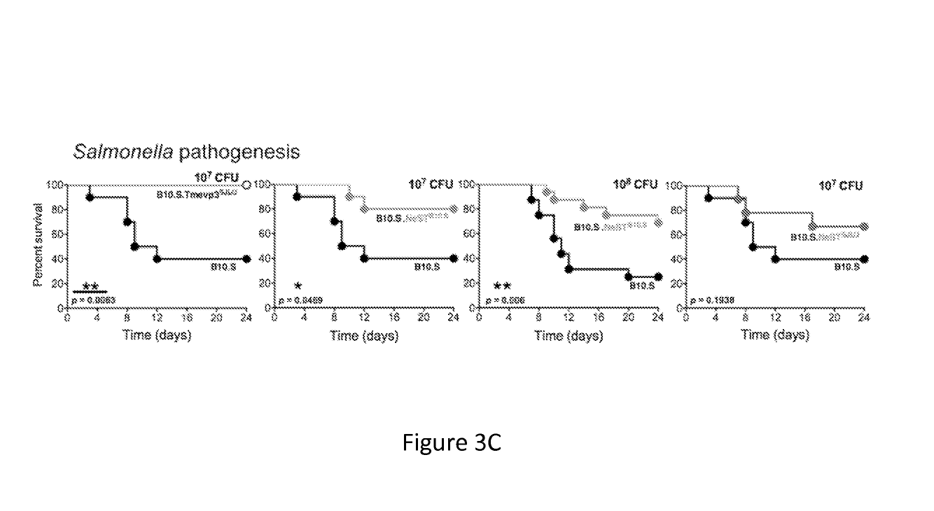

These analyses identified Il22, Ifng, and NeST as the most likely candidates for the gene or genes responsible for the Tmevp3 locus phenotypes by virtue of their polymorphic character and their known expression patterns. In FIG. 1C, the top and middle bar graphs represent the number of SNPs in a series of nonoverlapping 50 kb window regions. The regions of densest polymorphism between the congenic and parental lines can be seen in more detail in the bottom part of FIG. 1C. The product of Mdm1 is expressed predominantly in the retina (Chang et al. (2008) Hum. Mol. Genet. 17:3929-3941), making it an unlikely candidate. The three most polymorphic genes are Ifng, NeST, and Il22. The polymorphisms corresponding to NeST are shown in light gray, and all polymorphisms in the locus are listed in Table 1. We were especially interested in the lncRNA because of its potential novelty. As shown in FIG. 1D, CD3⁺ T cells from B10.S.Tmevp3^{SJL/J} mice displayed significantly higher amounts of NeST RNA than did those from B10.S mice. This result differs from that reported by Vigneau et al. (2003) J. Virol. 77:5632-5638), possibly due to differences in the T cell preparations used or the use of saturating RT-PCR methods in the previous study. Here, quantitative RT-PCR (qRT-PCR), the use of standard curves, and comparisons of RNA abundances from identical numbers of cells showed repeatedly that splenocytes from mice with an SJL/J-derived Tmevp3 allele accumulated substantially more NeST RNA than those from mice with a B10.S-derived Tmevp3 allele. Even so, the amount of NeST RNA that accumulated in total CD3⁺ T cells was, on average, only 0.15 molecules per cell (FIG. 1D). It is known that many lncRNAs are present at similarly low amounts but still are sufficient to cause epigenetic changes that are then self-propagating (reviewed in Guttman and Rinn (2012) Nature 482:339-346). It is also possible that NeST RNA is more abundant in a subset of the CD3⁺ T cells. Indeed, a higher abundance of NeST RNA was observed in CD8⁺ T cells (FIG. 3B) than in total CD3⁺ T cells (FIG. 1D).

Additional Pathogen Phenotypes for the Tmevp3 Locus

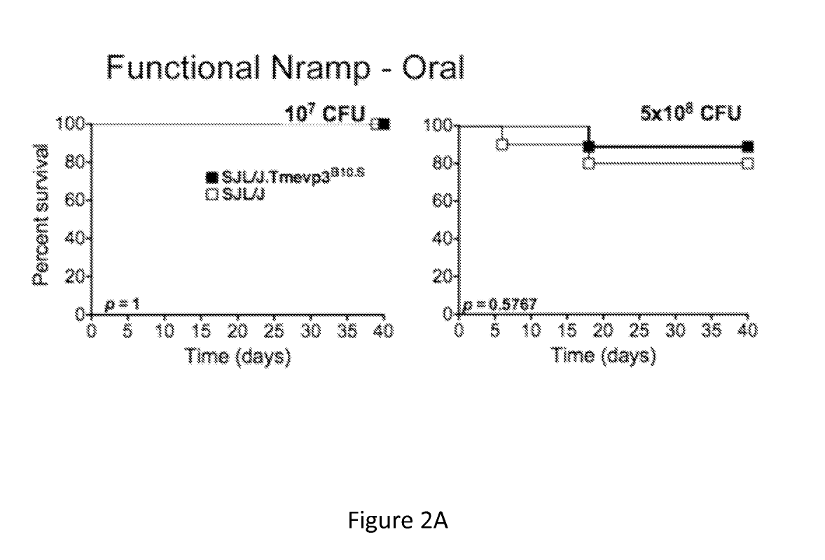

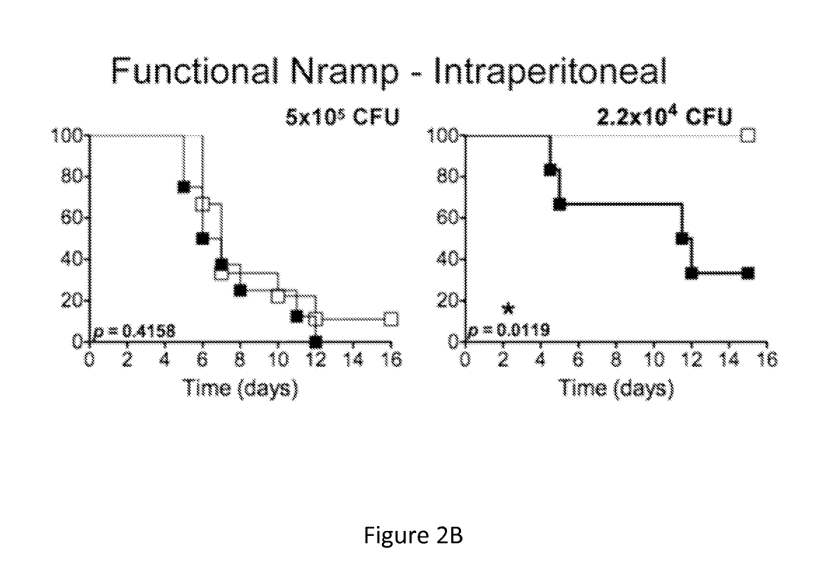

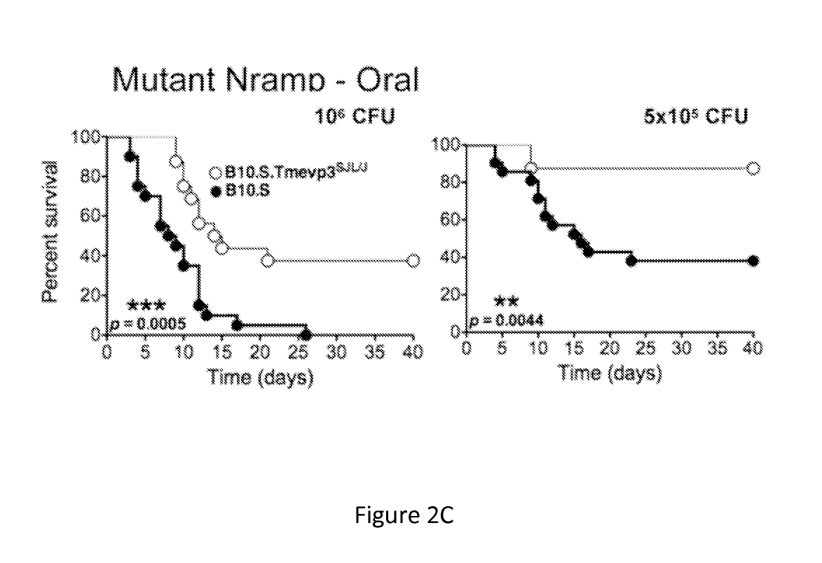

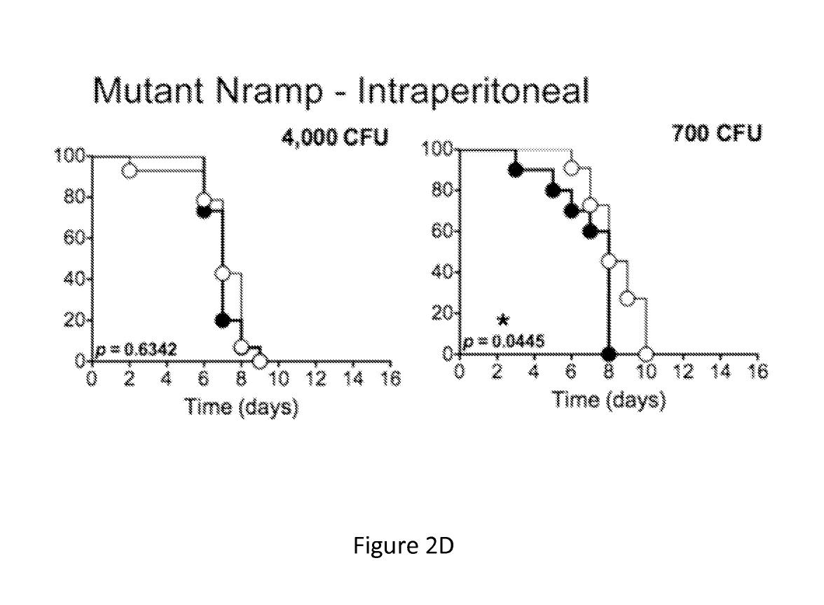

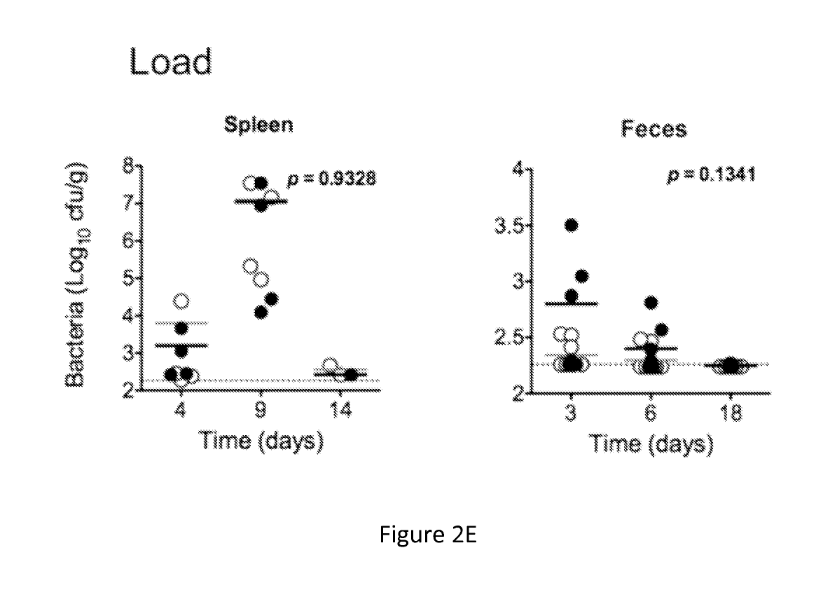

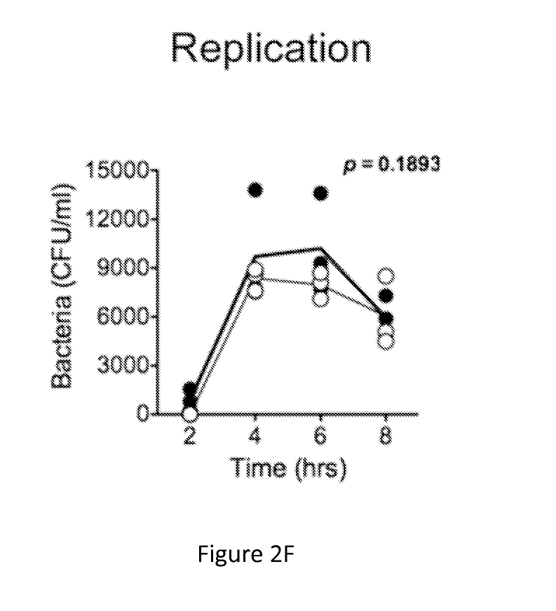

To determine whether Tmevp3 polymorphisms affected the outcome of another infection, we monitored their effects on the pathogenesis of Salmonella enterica Typhimurium, a pathogen that, like Theiler's virus, grows in macrophages and is extremely sensitive to IFN-γ and CD8⁺ T cell control (Monack et al. (2004) J. Exp. Med. 199:231-241; Rossi et al. (1997) J. Virol. 71:3336-3340; Foster et al. (2005) J. Interferon Cytokine Res. 25:31-42; Rodriguez et al. (2003) J. Virol. 77:12252-12265). We began by comparing SJL/J and SJL/J.Tmevp3B10.S mice because the size of the introgressed region was smaller in this pair than in the B10.S and B10.S.Tmevp3^{SJL/J} pair (FIG. 1B). Both SJL/J mice and SJL/J.Tmevp3^{B10.S} mice are homozygous for the functional allele of Nramp1, which encodes an ion channel that facilitates clearance of Salmonella (Frehel et al. (2002) Cell. Microbiol. 4:541-556). As expected, both strains were resistant to oral inoculation (FIG. 2A). However, when subjected to the more-potent intraperitoneal inoculation, both groups were susceptible but the SJL/J.Tmevp3^{B10.S} mice showed significantly more mortality (FIG. 2B).

B10.S and B10.S.Tmevp3SJL/J mice carry the Nramp1^{169Asp/169Asp} loss-of-function allele, which increases their susceptibility to Salmonella infection. When inoculated orally, B10.S mice displayed significantly more mortality than B10.S.Tmevp3^{SJL/J} mice at several infectious dosages (FIG. 2C). Intraperitoneal inoculation was rapidly lethal for both mouse strains (FIG. 2D). The differences in phenotypes between SJL/J and SJL/J.Tmevp3^{B10.S} and also between B10.S and B10.S.Tmevp3^{SJL/J} mice strengthen the hypothesis that the Tmevp3 polymorphisms initially discovered by analysis of Theiler's virus persistence have implications for general immune function. In subsequent experiments, we focused on B10.S and B10.S.Tmevp3^{SJL/J} mice and Salmonella pathogenesis, given that oral infection is the natural route.

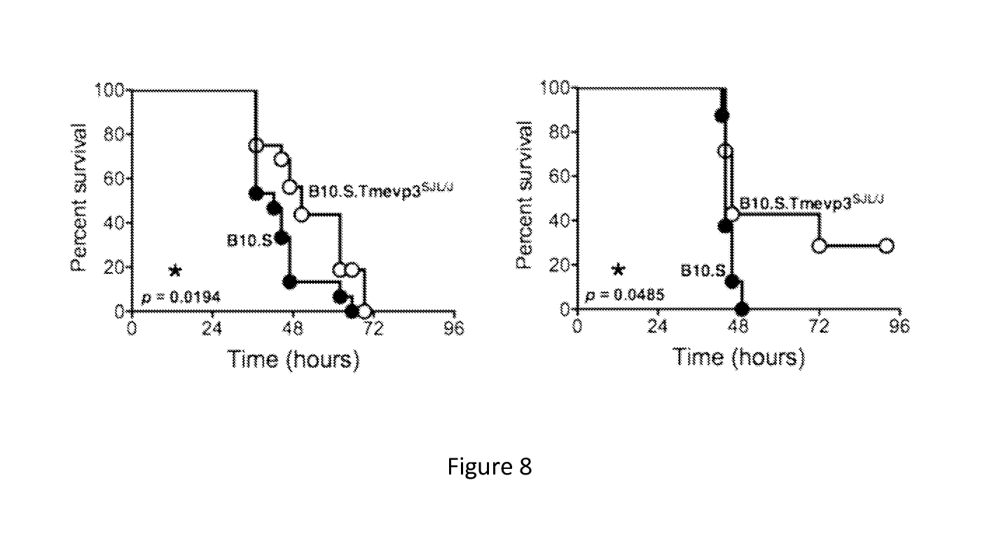

To determine whether the differences in phenotype resulted from different bacterial loads, we infected B10.S and B10.S. Tmevp3^{SJL/J} mice and monitored the abundance of S. Typhimurium in spleen and feces. B10.S and B10.S.Tmevp3SJL/J mice were orally inoculated with 10⁶ CFU and spleens were dissected 4, 9, and 14 days after inoculation. No significant differences in bacterial loads were observed in either spleen or feces at any time point (FIG. 2E). Interestingly, by day 14, both the B10.S and B10.S.Tmevp3^{SJL/J} mice had nearly resolved their infections even though mice from both groups continued to die. To test for differences in Salmonella growth in cultured macrophages, we infected bone-marrow-derived primary macrophages from B10.S and B10.S.Tmevp3^{SJL/J} and measured the amounts of intracellular Salmonella at various times after infection. No significant differences in bacterial growth within cells were observed (FIG. 2F). All of these data are consistent with the hypothesis that lethality is not due to the bacterial load per se, but rather to the inflammatory response to bacterial infection (Miao and Rajan (2011) Front. Microbiol. 2:85; Pereira et al. (2011) Front. Microbiol. 2: 33; Strowig et al. (2012) Nature 481:278-286). In fact, the SJL/J-derived Tmevp3 locus also conferred increased resistance to the lethal inflammatory disease caused by lipopolysaccharide (LPS) injection (FIG. 8).

Transgenic Expression of NeST RNA Reproduces the Phenotype Associated with the SJL/J Tmevp3 Allele

We hypothesized that NeST RNA could play a causal role in the phenotypes conferred by the Tmevp3 locus. To address this issue, we developed B10.S transgenic mice that express either SJL/J- or B10.S-derived NeST RNA under the control of a promoter that directs constitutive expression in both CD4⁺ and CD8⁺ T cells (FIG. 3A; Sawada et al. (1994) Cell 77:917-929). We obtained two transgenic mouse lines: B10.S.NeST^{B10.S} and B10.S.NeST^{SJL/J}. To test whether the transgenes had inserted near the endogenous Tmevp3 locus, we performed genetic crosses between the B10.S.NeST^{B10.S} and the B10.S.NeST^{SJL/J} transgenic mice and mice that bore neither marker. For both transgenic lines, the NeST transgenes and the endogenous locus showed no evidence of linkage (data not shown). Both transgenic NeST RNAs were expressed in CD8⁺ T cells (FIG. 3B), although at different amounts. The B10.S NeST transgene was expressed to an abundance similar to that of the endogenous NeST gene in the B10.S.Tmevp3^{SJL/J} line, whereas the SJL/J-derived transgene accumulated to much greater abundance (FIG. 3B).

To test whether the transgenic RNAs conferred protection against Salmonella pathogenesis, we inoculated B10.S mice, B10.S mice congenic at the Tmevp3 locus, and B10.S mice transgenic for each NeST allele orally with Salmonella. Mice that expressed the NeST B10.S transgene completely recapitulated the Tmevp3^{SJl/J} survival phenotype (FIG. 3C). Mice that expressed the SJL/J NeST transgene also showed protection. These findings demonstrate that NeST RNA can function in trans to reduce Salmonella pathogenesis.

Transgenic NeST RNA Expression Prevents Clearance of Theiler's Virus

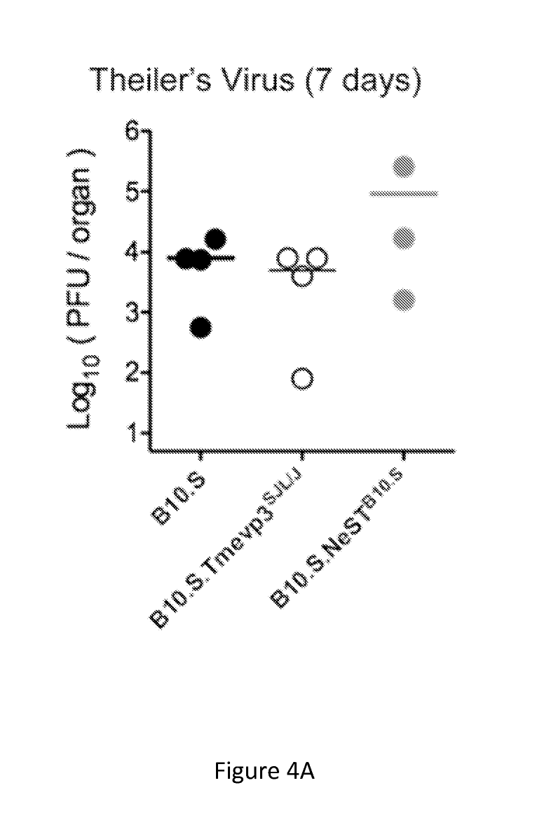

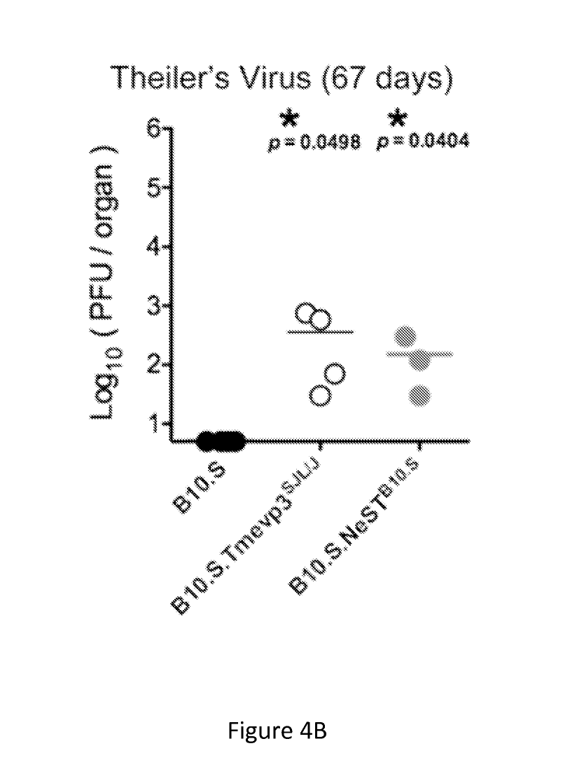

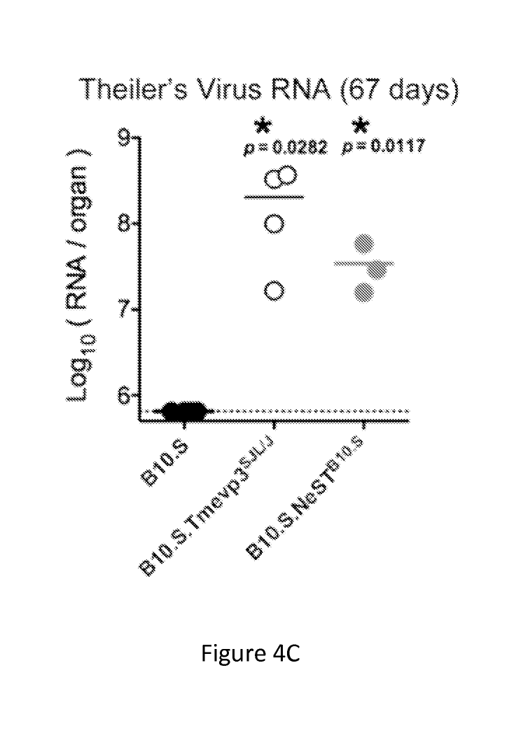

To test the role of NeST RNA in Theiler's virus infection, the microbial susceptibility phenotype that led to its discovery, we inoculated B10.S, B10.S.Tmevp3^{SJL/J}, and B10.S.NeSTB10.S transgenic mice by intracranial injection. Viral loads in the spinal cord were determined seven and 67 days after inoculation. At seven days, all strains displayed comparable viral titers (FIG. 4A), suggesting that NeST RNA plays little role during the acute phase of infection. However, 67 days after inoculation, infectious virus could only be recovered from mice that carried the NeST transgene or the B10.S.Tmevp3^{SJL/J} locus (FIG. 4B). The amounts of viral RNA in the spinal cords of the transgenic mice and the B10.S.Tmevp3^{SJL/J} mice were orders of magnitude higher than those found in the spinal cords of the nontransgenic B10.S parent (FIG. 4C). Thus, the susceptibility to Theiler's virus persistence in spinal cord associated with the Tmevp3SJL/J allele was recapitulated by the expression of the NeST RNA transgene.

Effect of the Tmevp3 Locus on the Expression of IFN-γ by CD8⁺ T Cells

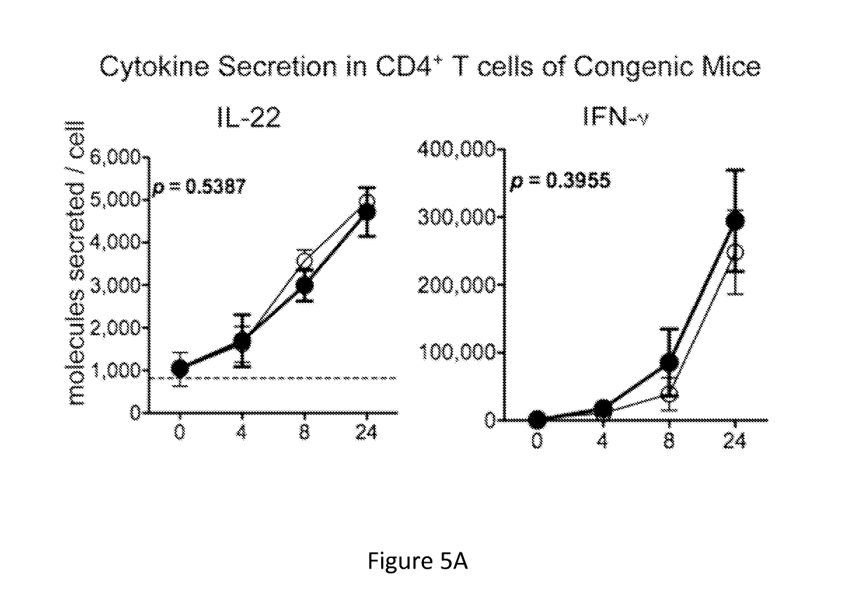

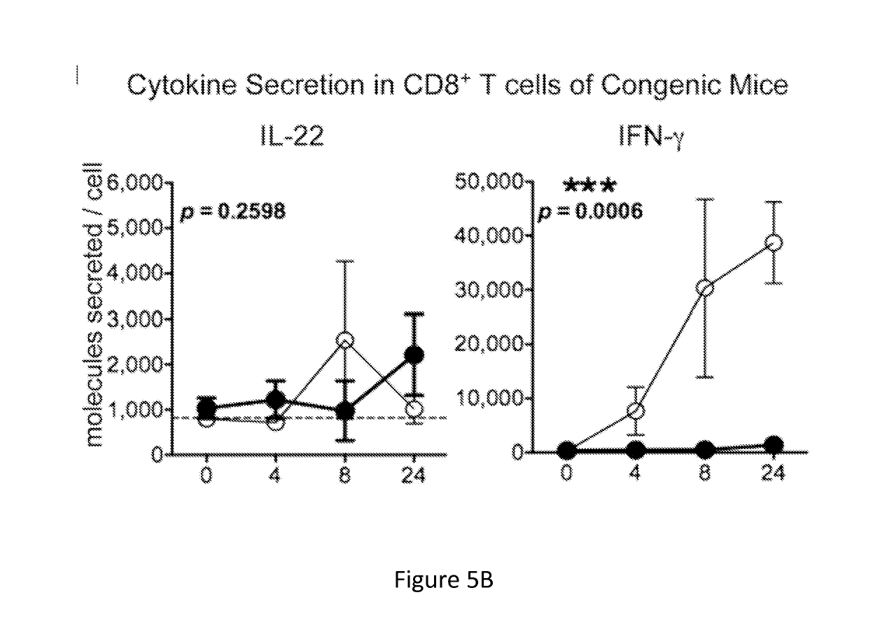

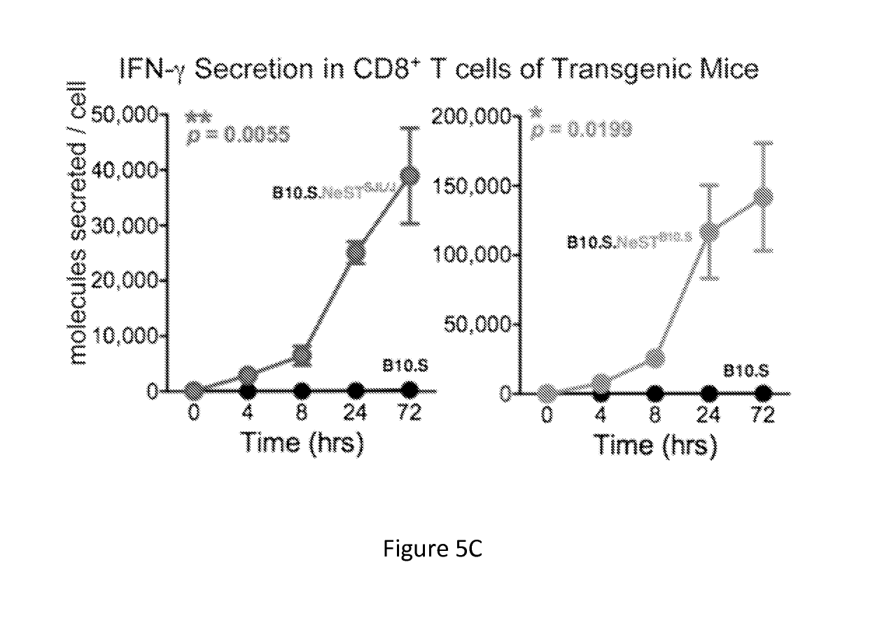

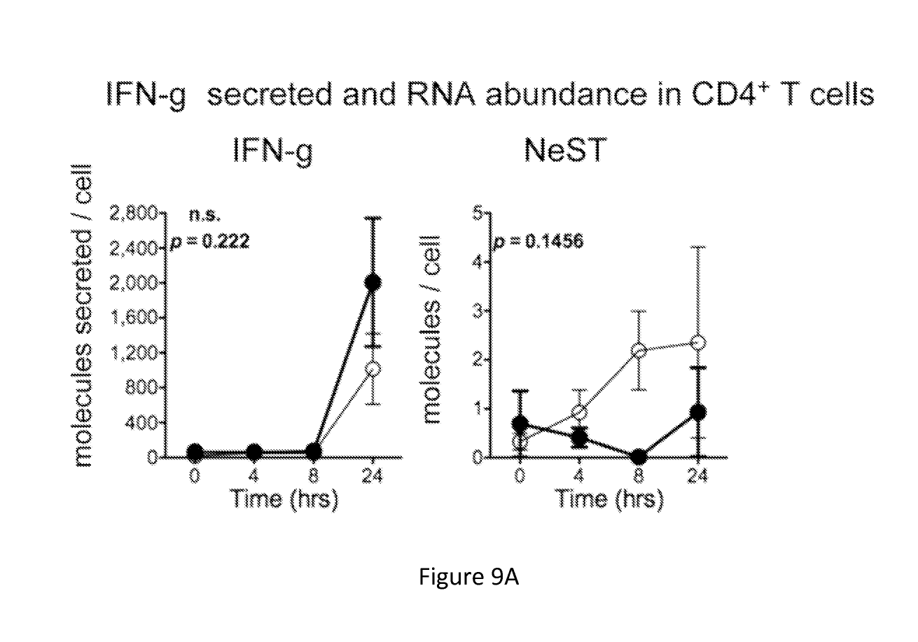

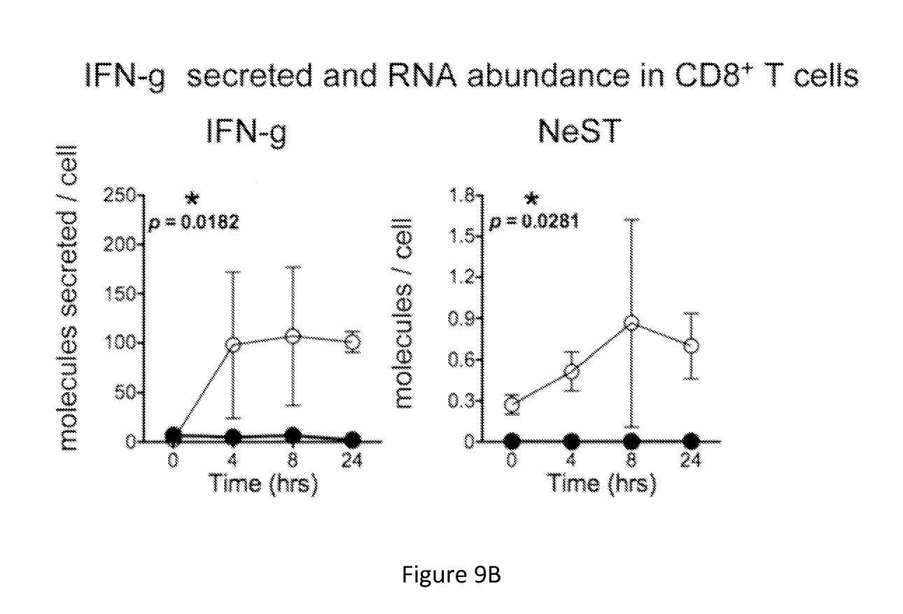

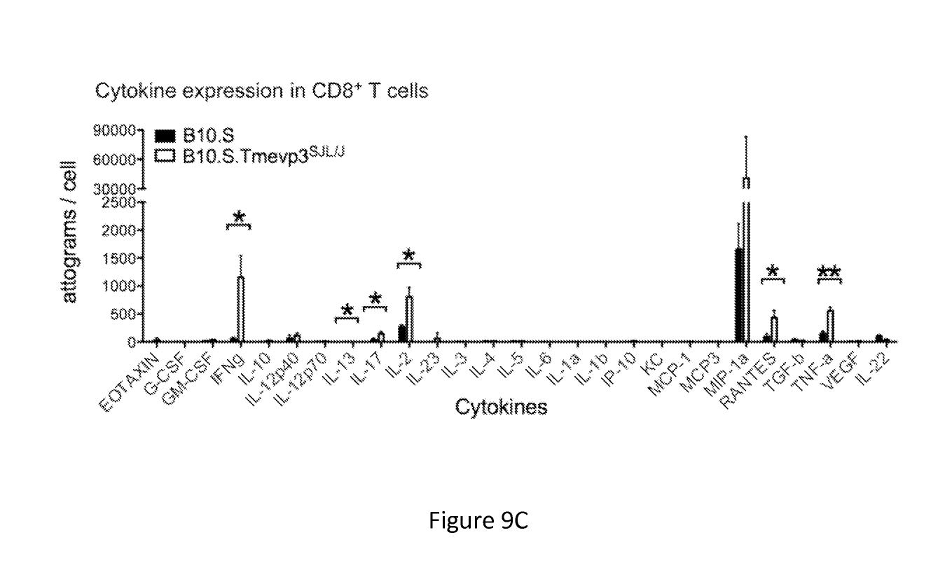

Several enhancer-like lncRNAs are known to activate neighboring genes, as exemplified by HOTTIP and Jpx (Ørom et al. (2010) Cell 143:46-58; Tian et al. (2010) Cell 143:390-403; Wang et al. (2011) Mol. Cell. 43:904-914). The physical proximity of Il22 and Ifng to NeST inspired us to test for differences in expression of these two genes in CD4⁺ and CD8⁺ T cells from B10.S and B10.S.Tmevp3^{SJL/J} mice. Isolated CD4⁺ and CD8⁺ T cells were cultured for 1 day, stimulated with phorbol 12-myristate 13-acetate (PMA) and ionomycin, and monitored for both cytokine secretion (FIGS. 5A and 5B) and intracellular RNA abundance (FIG. 9). In CD4⁺ T cells, ex vivo stimulation caused large but similar increases in the secretion of both cytokines in both B10.S and B10.S.Tmevp3^{SJL/J} mice (FIG. 5A). Similarly, the Tmevp3 allele did not significantly affect the amounts of IL-22 secreted from CD8⁺ T cells. However, whereas the amount of IFN-γ secreted from CD8⁺ T cells derived from B10.S mice was barely detectible, IFN-γ secretion from B10.S.Tmevp3^{SJL/J} mice was robust after stimulation (FIG. 5B). The difference in IFN-γ production by CD8⁺ T cells coincided with the amounts of IFN-γRNA and NeST RNA (FIG. 9B). These results show a strong correlation between the abundance of NeST RNA and IFN-γ RNA and the amount of IFN-γ protein in activated CD8⁺ T cells.

Transgenic Expression of NeST Induces IFN-g Synthesis in Activated CD8⁺ T Cells

To determine whether the expression of NeST RNA alone could elicit the observed changes in IFN-γ expression in CD8⁺ T cells, we monitored the abundance of the cytokine in CD8⁺ T cells from B10.S, B10.S.NeST^{SJL/J}, and B10.S.NeST^{B10.S} mice. As before, CD8⁺ T cells from the parental B10.S mice accumulated very little cytokine after ex vivo stimulation (FIG. 5C). However, the transgenic expression of either the B10.S or the SJL/J allele of NeST conferred the ability to induce IFN-γ secretion. Interestingly, the SJL/J-derived NeST RNA was less effective than the B10.S-derived RNA in mediating IFN-γ production, but both alleles caused statistically significant increases in IFN-γ expression upon CD8⁺ T cell activation. Subsequent experiments were designed to investigate the mechanism of these effects.

NeST is a Nuclear lncRNA that can Function in Trans to Affect its Neighboring Locus

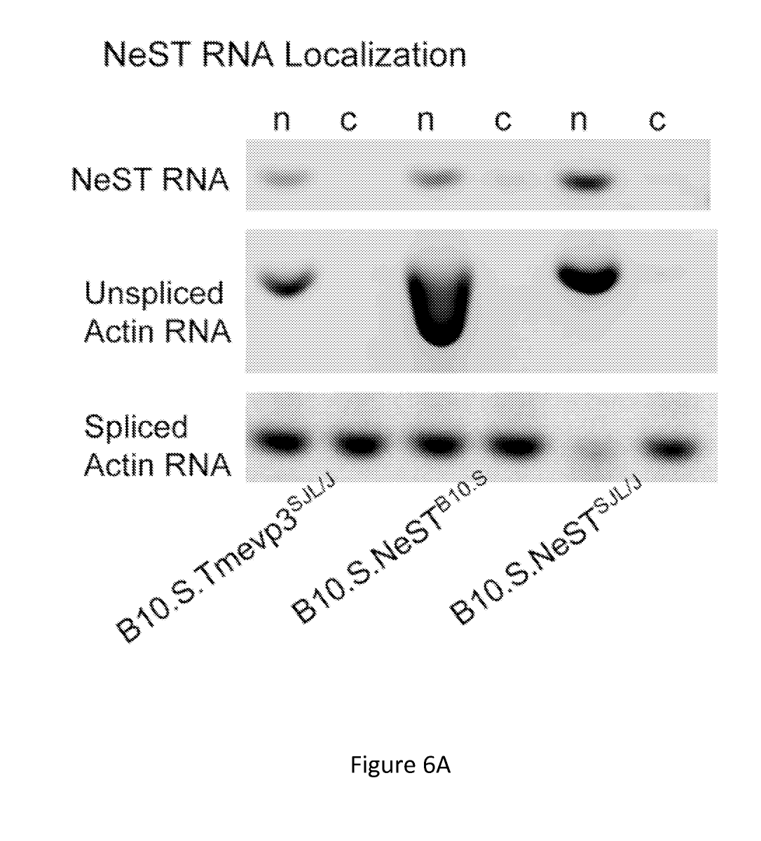

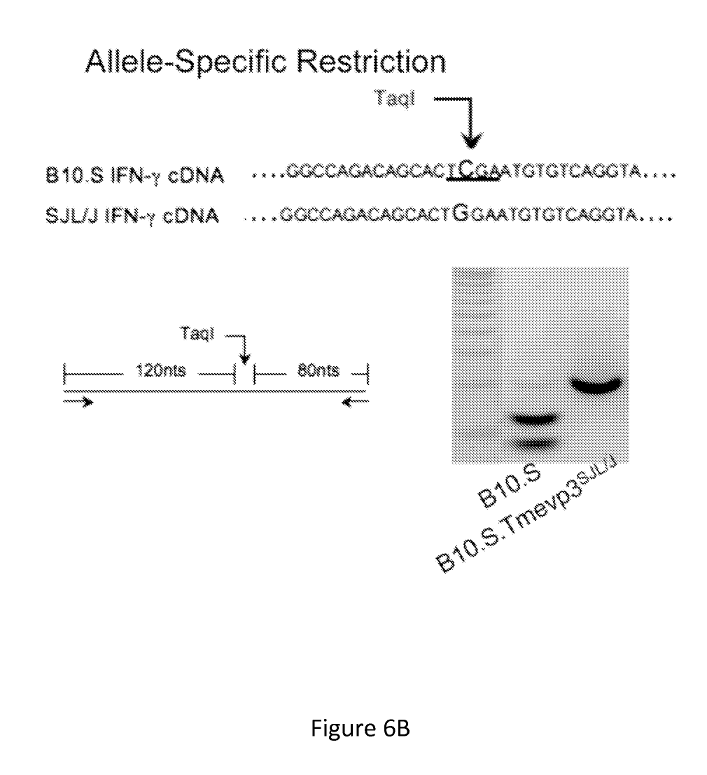

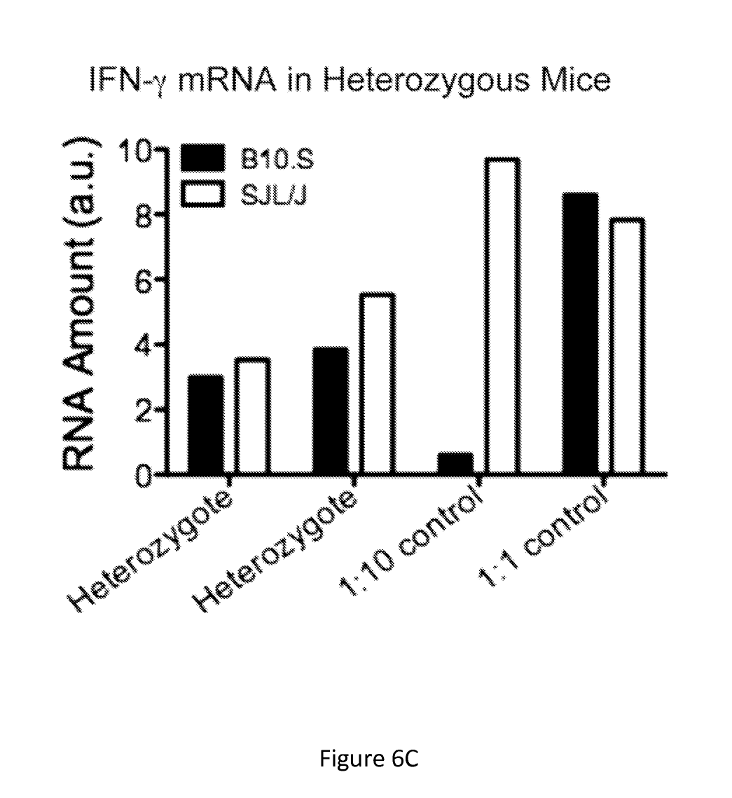

We hypothesized that, like several lncRNAs, NeST RNA affects IFN-γ accumulation at the transcriptional level by interacting with chromatin modification complexes. Consistent with this idea, most of the NeST RNA in either congenic or transgenic mice was found in the nuclear fraction of CD8⁺ T cells, cofractionating with unspliced (but not with spliced) actin mRNA (FIG. 6A).

The finding that two different transgenic NeST RNAs that were not genetically linked to the Ifng locus conferred the properties of the Tmevp3^{SJL/J} locus to B10.S mice (FIGS. 3, 4, and 5) argues that this lncRNA can function in trans. To determine whether NeST RNA can indeed function in trans from its normal position of synthesis, we took advantage of the fact that NeST RNA is expressed in stimulated CD8⁺ T cells of B10.S.Tmevp3^{SJL/J} mice but not in CD8⁺ T cells of B10.S mice (FIGS. 3B and S2B). We developed a PCR assay to distinguish between the SJL/J- and B10.S-derived IFN-γ alleles (FIG. 6B). CD8⁺ T cells from two heterozygous B10.S/B10.S.Tmevp3^{SJL/J} mice were stimulated, RNA was extracted, and the allele from which the RNA was transcribed was determined from the RT-PCR shown in FIG. 6B. Approximately equal amounts of IFN-γ mRNA from the B10.5 and SJL/J alleles accumulated following stimulation (FIG. 6C), arguing that the single functional NeST gene in the heterozygous mice could stimulate transcription from the Ifng genes on both chromosomes.

NeST RNA Binds to a Subunit of the MLL/SET1 H3K4 Methylase Complex and Increases Chromatin Modification at the Ifng Locus

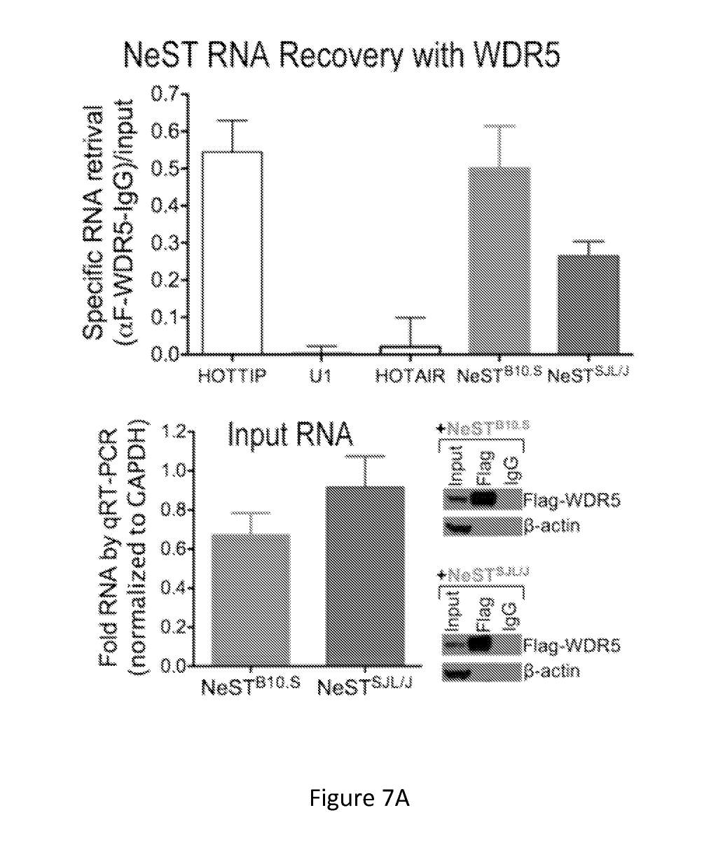

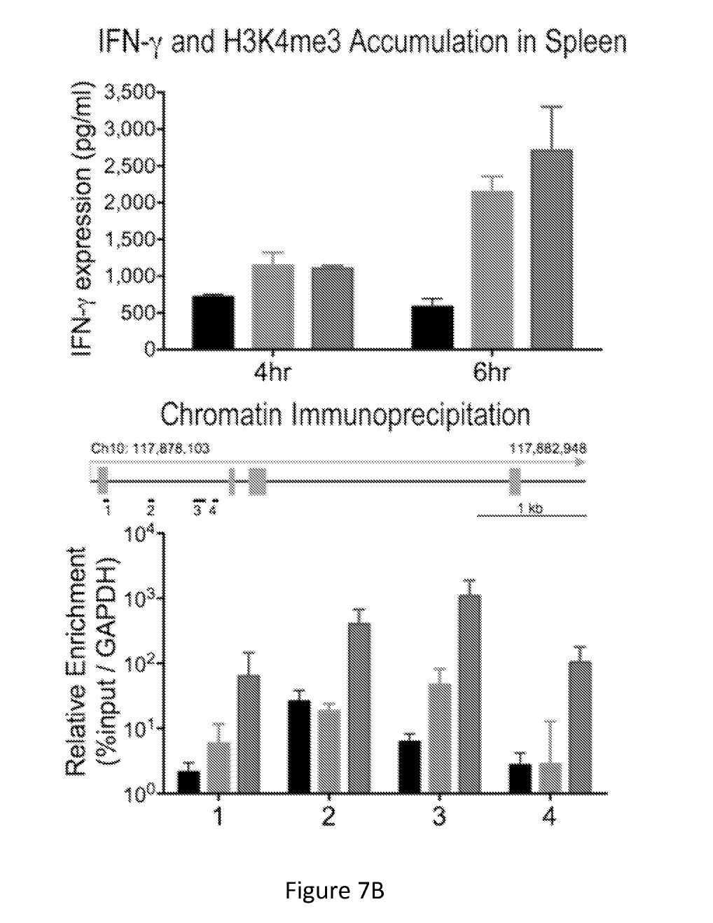

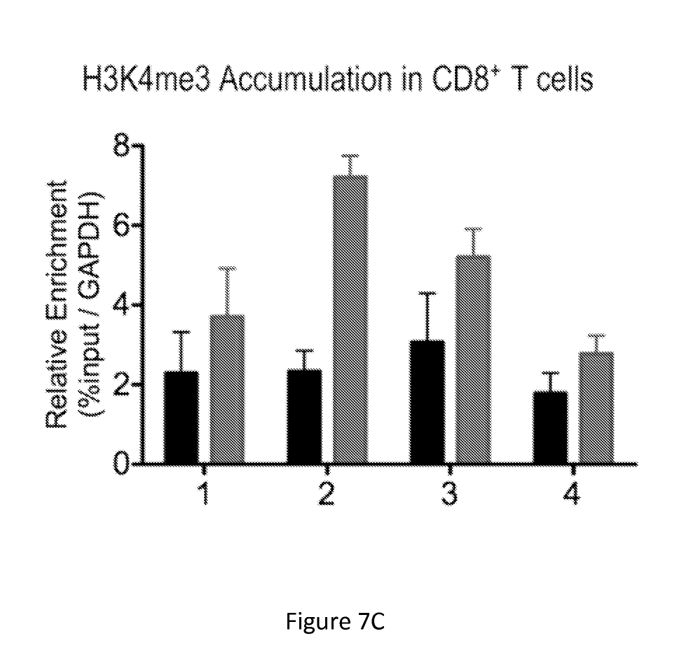

If NeST RNA were to have a direct effect on the expression of IFN-γ, via chromatin modification, it should be an activating effect. Recently, a new class of enhancer-like lncRNAs was discovered (Ørom et al., supra; Wang et al. (2011) Mol. Cell. 43:904-914). Among these, HOTTIP lncRNA was found to bind WDR5 protein to recruit complexes that facilitate transcription (Wang et al. (2011) Nature 472:120-124). WDR5 is a core subunit of MLL1-4 and SET1A/1B complexes, which catalyze the methylation of histone H3 at lysine 4, a mark of active gene expression. To test whether NeST RNA physically interacts with WDR5, the epitope-tagged protein was coexpressed in combination with a variety of RNAs via transient transfection of 293T cells (FIG. 7A). Extracts were then prepared, and WDR5 protein was immunoprecipitated and tested for associated RNAs by qRT-PCR. HOTTIP served as a positive control, and both HOTAIR lncRNA and U1 nuclear RNA served as negative controls. Immunoprecipitation of WDR5 specifically retrieved both NeST RNAs and HOTTIP, but not U1 or HOTAIR RNAs (FIGS. 7A and 7B). The physical interaction between NeST and WDR5 raises the intriguing possibility that NeST may control H3K4 methylation at the Ifng locus.

To examine the contribution of NeST RNA to IFN-γ production during immune challenge, we used a well-characterized mouse model of sepsis: intraperitoneal injection of LPS. B10.S mice as well as B10.S.NeST^{B10.S} and B10.S.NeST^{SJL/J} transgenic mice were injected with LPS. By 6 hours postinjection, the presence of either transgenic NeST allele increased the amount of IFN-γ in splenic tissue compared with the B10.S control (FIG. 7B). An increase in H3K4me3 occupancy at the Ifng locus preceded this increased IFN-γ synthesis by 2 hours (FIG. 7B). Transgenic mice with the SJL/J-derived allele, which accumulate much more NeST RNA than those that express the B10.S allele (FIG. 3B), showed a larger amount of IFN-γ-encoding DNA with H3K4me3 modifications (FIG. 7B). Thus, increased NeST RNA abundance can result in more extensive H3K4me3 modification. Still, NeST RNA is extremely potent even at low abundance, either because the epigenetic effects persist in its absence or because activation of only a subset of cells is necessary for the observed phenotypes.

The high occupancy of H3K4me3 at the Ifng locus in the B10.S.NeST^{SJL/J} transgenic mice allowed us to measure chromatin modification in isolated primary CD8⁺ cells in the presence and absence of NeST RNA. Following activation of B10.S- and B10.S.NeST^{SJL/J}-derived CD8⁺ T cells, we found that the presence of NeST^{SJL/J} RNA caused an increase in H3K4me3 at the Ifng locus (FIG. 7C). The NeST RNA-dependent increase in H3K4me3 activation in both total splenic cells and CD8⁺ T cells strongly suggests that, by binding to WDR5, NeST RNA is required to program an active chromatin state that confers inducibility to the Ifng gene.

Discussion

In this work, we performed a genetic analysis of an lncRNA expressed in T cells. Mice that express NeST RNA, either in its natural chromosomal environment or by transgenic delivery, displayed increased resistance to Salmonella-induced pathogenesis but increased susceptibility to Theiler's virus persistence. These disparate effects illustrate the role of balanced polymorphisms in susceptibility to infectious disease (Dean et al. (2002) Annu Rev. Genomics Hum. Genet. 3:263-292; Liu et al. (1996) Cell 86:367-377; Williams et al. (2005) Nat. Genet. 37:1253-1257; Wang et al. (2010) Hum. Mol. Genet. 19:2059-2067; Cagliani et al. (2011) BMC Evol. Biol. 11:171). Genes of the immune system are under purifying selection by challenges from a plethora of pathogens, and mutations that protect against one microbe may increase susceptibility to another. In the case of autoimmunity, the rs2076530-G allele of BTNL2, a major histocompatibility complex (MHC) II-linked gene, confers increased susceptibility to rheumatoid arthritis and type 1 diabetes but decreased susceptibility to multiple sclerosis and autoimmune thyroiditis (Orozco et al. (2005) Hum. Immunol. 66:1235-1241; Sirota et al. (2009) PLoS Genet. 5:e1000792; Valentonyte et al. (2005) Nat. Genet. 37:357-364).

A potential explanation for the disparate effects of NeST RNA on Theiler's virus persistence and Salmonella pathogenesis is that it alters the magnitude or timing of the inflammatory responses. CD8⁺ T cell populations are extremely heterogeneous (Davila et al. (2005); Joosten et al. (2007) Proc. Natl. Acad. Sci. USA 104:8029-8034; Xystrakis et al. (2004) Blood 104:3294-3301); for example, the CD8⁺ T_{reg} population is important in resolving inflammation and preventing autoimmunity (Frisullo et al. (2010) Hum. Immunol. 71:437-441; Sun et al. (2009) Nat. Med. 15:277-284; Trandem et al. (2011) J. Immunol. 186:3642-3652). Alternatively, NeST-dependent activation of basal inflammation could serve to attenuate subsequent inflammatory events. Finally, NeST RNA may have targets in addition to the Ifng gene that contribute to its apparently anti-inflammatory effect (FIG. 9).

The fact that the effects of NeST can be conferred by transgenic expression from ectopic loci, and to Ifiig alleles on both chromosomes when NeST is expressed heterozygously, argues that NeST function, even on the adjacent IFN-γ-encoding locus, can be provided in trans. Although many lncRNAs, such as Xist and HOTTIP, exert their function on neighboring genes exclusively in cis, trans-acting lncRNA function has precedent in HOTAIR, linc-p21, and Jpx lncRNAs (reviewed in Guttman and Rinn, supra). Notably, Jpx is required to activate the expression of the adjacent Xist gene on the presumptive inactive X chromosome, and this activation can occur whether Jpx RNA is supplied in cis or trans (Tian et al. (2010) Cell 143:390-403). Thus, there is increasing recognition in the field that lncRNA regulation of nearby genes can occur by trans-acting mechanisms. The increased demands made on these lncRNAs for target specificity are currently under investigation.

In the vicinity of the Ifng locus, many of the distal regulatory elements map to regions now known to encode NeST (Sekimata et al. (2009) Immunity 31:551-564). For example, acetylation of histone 4 (H4Ac), a mark of active transcription, has been observed in discrete regions surrounding Ifng in activated CD4⁺ and CD8⁺ T cells. One peak in particular, which correlates well with the differentiation of both CD8⁺ and CD4⁺ T cells (Chang and Aune (2005) Proc. Natl. Acad. Sci. USA 102:17095-17100; Zhou et al. (2004) Proc. Natl. Acad. Sci. USA 101:2440-2445), is located 59 kb downstream of Ifng and coincides with the sixth exon of NeST (FIG. 1). Another noteworthy region that is critical for IFN-γ expression in CD4 T cells maps 66 kb downstream of murine Ifng and, in humans, 166 kb downstream of IFNG. This regulatory element is also located in the NeST gene. It contains a lineage-specific DNase I hypersensitive site found in Th1 but not Th2 CD4 T cells (Balasubramani et al. (2010) Immunol. Rev. 238:216-232). During lineage-specific induction of IFN-γ, the proteins CTCF, T-bet, and cohesin all localize to these sequences. Indeed, a recent study (Collier et al. (2012) J. Immunol. 189:2084-2088) related the expression of NeST RNA (Tmevpg1) to the expression of IFN-γ in CD4⁺ T cells by a mechanism that depends on the simultaneous expression of T-bet. Simultaneous binding of cohesin, T-bet, and CTCF results in a complex three-dimensional conformation that is predicted to bring the NeST and IFN-γ coding regions into direct proximity (Hadjur et al. (2009) Nature 460:410-413; Ong and Corces (2011) Nat. Rev. Genet. 12:283-293; Sekimata et al. (2009) Immunity 31:551-564).

Humans express an RNA species homologous to NeST that also appears to be noncoding and is transcribed adjacent to the IFNG locus from the opposite DNA strand. Interestingly, polymorphisms that correlate with autoimmune and inflammatory diseases such as rheumatoid arthritis, Crohn's disease, and multiple sclerosis have been found in the DNA sequence that encodes the first intron of the IFN-γ-encoding gene (Goris et al. (2002) Immun. 3:470-476; Latiano et al. (2011) PLoS ONE 6:e22688; Silverberg et al. (2009) Nat. Genet. 41:216-220); this DNA region also encodes the fifth intron of the overlapping NeST RNA-encoding gene. As they do in mice, variations in NeST RNA expression in humans could contribute to differences in T cell response and disease susceptibility. Whether and how disease-associated SNPs alter human NeST expression or function will be addressed in future studies.

Natural polymorphisms, both in humans and in animal models, can yield subtle quantitative allelic effects that are more difficult to study but are more relevant to human medicine than the effects of gene knockout or other loss-of-function genetic techniques. The discovery of NeST RNA was the result of classical forward genetics. Our analysis of NeST RNA was based on the conceptual framework that regions that are thought to be “intergenic” encode functional RNA elements. Many genome-wide association studies have also pointed to intergenic regions as heritable causes of human disease (Libioulle et al. (2007) PLoS Genet. 3:e58; Sotelo et al. (2010) Proc. Natl. Acad. Sci. USA 107:3001-3005). This study establishes that some of these intergenic regions may encode functional lncRNAs that are critical for proper gene regulation. The promise of individualized medicine relies on our understanding of as many genetic polymorphisms as possible in the context of individual immunological and other environmental experiences.

While the preferred embodiments of the invention have been illustrated and described, it will be appreciated that various changes can be made therein without departing from the spirit and scope of the invention.

What is claimed is:

1. A method of modulating an immune response in a subject, the method comprising administering a therapeutically effective amount of NeST or a NeST inhibitor to the subject.

2. The method of claim 1, wherein NeST increases production of interferon-gamma (IFN-γ) by leukocytes, whereby CD4+ T helper (Th) cells, CD8+ cytotoxic T cells, or macrophages are activated in the subject.

3. The method of claim 2, wherein NeST is provided by a recombinant polynucleotide comprising a promoter operably linked to a polynucleotide encoding NeST.

4. The method of claim 1, wherein the subject has an infectious disease.

5. The method of claim 1, wherein the infectious disease is caused by an intracellular pathogen.

6. The method of claim 5, wherein the infectious disease is caused by a virus.

7. The method of claim 6, wherein the virus is selected from the group consisting of influenza virus, respiratory syncytial virus, hepatitis virus B, hepatitis virus C, herpes virus, papilloma virus, and human immunodeficiency virus.

8. The method of claim 4, wherein the infectious disease is caused by a bacterial infection.

9. The method of claim 8, wherein the bacterial infection is antibiotic-resistant.

10. The method of claim 8, wherein the infectious disease is tuberculosis, listeriosis, diphtheria, food poisoning, or sepsis.

11. The method of claim 4, wherein the infectious disease is caused by a fungal infection.

12. The method of claim 11, wherein the infectious disease is selected from the group consisting of aspergillosis, blastomycosis, and candidosis.

13. The method of claim 4, wherein the infectious disease is caused by a parasite.

14. The method of claim 13, wherein the infectious disease is selected from the group consisting of malaria, leishmaniasis, toxoplasmosis, schistosomiasis, and clonorchiasis.

15. The method of claim 1, wherein the subject has cancer or tumors.

16. The method of claim 1, further comprising administering a vaccine to the subject.

17. The method of claim 1, wherein the subject has chronic granulomatous disease, congenital osteopetrosis, idiopathic pulmonary fibrosis, ovarian cancer, bladder carcinoma, systemic sclerosis, or tuberculosis.

18. The method of claim 1, wherein a NeST inhibitor selected from the group consisting of a small interfering RNA (siRNA), a microRNA (miRNA), a Piwi-interacting RNA (piRNA), a small nuclear RNA (snRNA), and an antisense oligonucleotide is administered to the subject.

19. The method of claim 18, wherein the NeST inhibitor reduces inflammation in the subject.

20. The method of claim 18, wherein the subject has an inflammatory condition or an autoimmune disorder.

21. The method of claim 20, wherein the subject has multiple sclerosis, rheumatoid arthritis, stomatitis, lupus erythematosus, ischemic heart disease, atherosclerosis, cancer, fibrosis, autoimmune thyroid disease (AITD), inflammatory bowel disease, inflammatory myopathy, giant cell arteritis (GCA), asthma, allergy, Parkinson's disease, or Alzheimer's disease.

22. The method of claim 18, wherein the subject has damaged tissue or a wound.

23. The method of claim 1, wherein the subject is a human.

24. A method of increasing production of interferon-gamma (IFN-γ) by leukocytes in a subject, the method comprising administering an effective amount of NesT to the subject.

25. The method of claim 24, wherein CD4+ T helper (Th) cells, CD8+ cytotoxic T cells, or macrophages are activated in the subject.

26. The method of claim 24, wherein the subject has cancer or tumors.

27. The method of claim 24, wherein the subject has an infection by a pathogen.

28. The method of claim 27, wherein the pathogen is a virus, bacterium, protist, or fungus.

29. The method of claim 24, wherein the subject is immunodeficient.

30. The method of claim 24, wherein NeST is provided by a recombinant polynucleotide comprising a promoter operably linked to a polynucleotide encoding NeST.

31. A method of decreasing production of interferon-gamma (IFN-γ) by leukocytes in a subject, the method comprising administering an effective amount of a NeST inhibitor to the subject.

32. The method of claim 31, wherein the NeST inhibitor is selected from the group consisting of a small interfering RNA (siRNA), a microRNA (miRNA), a Piwi-interacting RNA (piRNA), a small nuclear RNA (snRNA), and an antisense oligonucleotide.

33. The method of claim 31, wherein the NeST inhibitor is provided by a recombinant polynucleotide comprising a promoter operably linked to a polynucleotide encoding a NeST inhibitor.

34. The method of claim 31, wherein the subject shows reduced inflammation after treatment.

35. The method of claim 31, wherein the subject has an inflammatory condition or autoimmune disorder.

36. The method of claim 35, wherein the subject has multiple sclerosis, rheumatoid arthritis, stomatitis, lupus erythematosus, ischemic heart disease, atherosclerosis, cancer, fibrosis, autoimmune thyroid disease (AITD), inflammatory bowel disease, inflammatory myopathy, giant cell arteritis (GCA), asthma, allergy, Parkinson's disease, or Alzheimer's disease.

37. The method of claim 35, wherein the subject has damaged tissue or a wound.


SEQUENCE LISTING (25 sequences)

SEQ ID NO: 1 - DNA, 1791 bp, Homo sapiens
ctgcaatttcaggtagcttttctgactcttaaagagatctcaagtataccttcagagaaatgccagcaaaaactgtagtcatttgggaaggaataagcctggaagaaaaagatacaacgaactagcacaacgaggagtttgaaaagttcatgacagctcacagctgatgatggtggcaatcttaaggatacagaaagctcattcctcatgcagggaagaagaaaatattctaaagaagagataagcatattccatgaaatcaaaaaagcgtaaaacgctggaggagaagtcagtagcaagcagctaagacaacatggtacatgtggctagaagcccaccaactgctaacaaccagctggagggtaagtcaaaagctatgagagcccagaaggagaacaattatttagacctggaggggccagttgggaaagtctttgaagggtcttcagaaaatggaccaaaagaaaagaggaaaagtcattctactgagaggtgtttgtgtaatcaaagcaaccgaggcctaagaactcacaaaagattagaaaagggtaaaggatgcctggaaagccggactaggaaaaggctgtgaagggccctgaaggtcacatgtcacatgttgcctgcagcatgcagaatcaaaacatgataaatcatgtcttgaatcatgacatatgaagcatgacacacgaatcatgacatgtgacatgttgaatgtcacatgtcacatgccgtaggcagccatcaagagtatttgaaaggaaagtgtataatcaggcctgtgctttagtaagacaatttgtatagtgttcagtgtggattaaaatggagaacacaggtatcagtttgacatcgacagtacaggaaaccatttgggtttcttgtacaccaaaatattaaaacatacacgcaaacacaccaacacattaaaatcttgagagagagggccaaaagttctttgtcgctggactaaaagtggaaataaatgtttacaacctctaaggtggattaagctaagaatttggagttgttggcaaaactgccacataaaatttcagacatgcaaagaaatgcaatcgtagacttatgcacatacttccaccagagaagggaatctgggcagagtgttcaaagcttaagaaattcctttgctcttcaccaagaagagccaacagagttttcgttgaatcaactcttatattcccttaaaaagaagttgatagtgttgtggaaaactcacccattgaagtgacaacaaaaacagcaacccagtatgagttaactggtattatgttaaagggacaagtaactgcacttttgacacacatcctctaaaaggtagggtgggagtgtttcaaagaaccaagtctgagtaatagaaaagcatataccacttccccatatacacgttcgagcatgggggagcaggaagctgggtaattgaatgcagttgtgcacactggcaattttcccgccacataccatttcttcggtgtctctcccaaaattctcctcctaagtggcacatccctttattgatcatctttccccatactgatcatcctcagcaatcatgtgagttattagtctaagtatttgtttttctcaaatatggtcttttaactactccctaaatattttatatatacatagtttgttttcccaatttaactacaagcatttaaaaaatgggaatatttgtctatatttctgtaatttttcatcatgccttatatggtatataaagatcaacagcaataaaacaacaacagtaataatagctaagaccttttg

SEQ ID NO: 2 - DNA, 20 bp, Artificial Sequence
  HOTTIP PCR primer
caaactccgtcctccaaaac

SEQ ID NO: 3 - DNA, 20 bp, Artificial Sequence
  HOTTIP PCR primer
cagtgaagagcgatcagtgg

SEQ ID NO: 4 - DNA, 20 bp, Artificial Sequence
  U1 PCR primer
atacttacctggcaggggag

SEQ ID NO: 5 - DNA, 21 bp, Artificial Sequence
  U1 PCR primer
cagggggaaagcgcgaacgca

SEQ ID NO: 6 - DNA, 25 bp, Artificial Sequence
  GAPDH PCR primer
aggtggaggagtgggtgtcgctgtt

SEQ ID NO: 7 - DNA, 22 bp, Artificial Sequence
  GAPDH PCR primer
ccgggaaactgtggcgtgatgg

SEQ ID NO: 8 - DNA, 22 bp, Artificial Sequence
  NeST PCR primer
caacgtacgctgcctcccgatg

SEQ ID NO: 9 - DNA, 23 bp, Artificial Sequence
  NeST PCR primer
ctatttggtcgagtctgacagag

SEQ ID NO: 10 - DNA, 22 bp, Artificial Sequence
  Ifng PCR primer
cctgttactacctgacacattc

SEQ ID NO: 11 - DNA, 23 bp, Artificial Sequence
  Ifng PCR primer
cctttacttcactgaccaataag

SEQ ID NO: 12 - DNA, 20 bp, Artificial Sequence
  Il-22 PCR primer
agaacgtcttccagggtgaa

SEQ ID NO: 13 - DNA, 19 bp, Artificial Sequence
  Il-22 PCR primer
gctacctgatgaaagcagg

SEQ ID NO: 14 - DNA, 20 bp, Artificial Sequence
  Actin PCR primer
gcctcgtcacccacatagga

SEQ ID NO: 15 - DNA, 20 bp, Artificial Sequence
  Actin PCR primer
aggtgtgatggtgggaatgg

SEQ ID NO: 16 - DNA, 22 bp, Artificial Sequence
  TMEV PCR primer
cccagtcctcaggaaatgaagg

SEQ ID NO: 17 - DNA, 22 bp, Artificial Sequence
  TMEV PCR primer
tccaaaaggagaggtgccatag

SEQ ID NO: 18 - DNA, 20 bp, Artificial Sequence
  Ifng1 PCR primer
ccatcggctgacctagagaa

SEQ ID NO: 19 - DNA, 20 bp, Artificial Sequence
  Ifng1 PCR primer
atgaggaagagctgcaaagc

SEQ ID NO: 20 - DNA, 20 bp, Artificial Sequence
  Ifng2 PCR primer
accaaaactacgcagggaaa

SEQ ID NO: 21 - DNA, 20 bp, Artificial Sequence
  Ifng2 PCR primer
gctggctttgattcgattgt

SEQ ID NO: 22 - DNA, 20 bp, Artificial Sequence
  Ifng3 PCR primer
tcagaggcctggaccataag

SEQ ID NO: 23 - DNA, 20 bp, Artificial Sequence
  Ifng3 PCR primer
gaaactgcaaggccacaaat

SEQ ID NO: 24 - DNA, 20 bp, Artificial Sequence
  Ifng4 PCR primer
atttgtggccttgcagtttc

SEQ ID NO: 25 - DNA, 20 bp, Artificial Sequence
  ; Ifng4 PCR primer
gggcccttccacttacttct